(19) United States
(12) Patent Application Publication
(10) Pub. No.: US 2013/0189757 A1
(43) Pub. Date: Jul. 25, 2013

(54) AFFINITY PURIFICATION OF RNA UNDER NATIVE CONDITIONS BASED ON THE LAMBDA BOXB/N PEPTIDE INTERACTION

(71) Applicant: Pascale Legault, Mont-Royal (CA)

(72) Inventor: Pascale Legault, Mont-Royal (CA)

(73) Assignee: VALORISATION-RECHERCHE, LIMITED PARTNERSHIP, MONTREAL, QC (CA)

(21) Appl. No.: 13/810,551

(22) PCT Filed: Jun. 30, 2011
(86) PCT No.: PCT/CA11/50400

§ 371 Date: Jan. 16, 2013

Related U.S. Application Data
(60) Provisional application No. 61/365,005, filed on Jul. 16, 2010.

(51) Int. Cl. C07H 1/06

(52) U.S. Cl. CPC C07H 1/06
USPC 435/188; 536/23.2; 435/270

(57) ABSTRACT

Reagents, methods, constructs and kits are described for immobilizing or purifying a target RNA of interest, based on the interaction of boxB RNA with a bacteriophage N peptide, which in turn is linked to an immobilizing moiety capable of binding to a solid support.

### CROSS REFERENCE TO RELATED APPLICATIONS

The present application claims the benefit of U.S. provisional Ser. No. 61/365,005, filed on Jul. 16, 2010, which is incorporated herein by reference in its entirety.

### TECHNICAL FIELD

The present invention generally relates to reagents and methods for nucleic acid-based applications, such as RNA immobilization and purification.

### BACKGROUND ART

Several recent discoveries have emphasized the importance of RNA-based processes in biology and have brought RNA molecules to the forefront of basic and applied biomedical research. As a result, there has been an increased demand to quickly generate large amounts of RNAs that are chemically pure and folded in their native conformation for biochemical, biophysical and structural studies. The traditional approach to purify RNA produced from in vitro transcription relies on denaturing polyacrylamide gel electrophoresis. Since this methodology involves denaturation of the RNA molecule (Uhlenbeck, O. C. (1995) RNA, 1, 4-6), it often results in RNA contaminated with acrylamide oligomers that are difficult to remove (Lukaysky, P. J. and Puglisi, J. D. (2004) RNA, 10, 889-893). This procedure is also generally very time-consuming and tedious.

Alternative purification methods have recently been developed to purify RNA in a time-efficient manner and under non-denaturing conditions. Examples include size-exclusion and ion-exchange chromatography (Shields, T. P., et al. (1999) RNA, 5, 1259-1267; Lukaysky, P. J. and Puglisi, J. D. (2004), supra; Kim, I. et al. (2007) RNA, 13, 289-294; McKenna, S. A., et al. (2007) Nature Protocols, 2, 3270-3277; Keel, A. Y. et al. (2009), Methods in Enzymology, Vol. 469, pp. 3-25; Easton, L. E. et al. (2010) RNA, 16, 647-653) and affinity purification (Cheong, H. K., et al. (2004) Nucleic Acids Res, 32, e84; Kieft, J. S. and Batey, R. T. (2004) RNA, 10, 988-995; Batey, R. T. and Kieft, J. S. (2007) RNA, 13, 1384-1389; Boese, B. J. et al. (2008). Nucleosides Nucleotides & Nucleic Acids, 27: 949-966; Pereira, M. J. et al. (2010), Plos One 5, e12953).

However, at this time, only a few procedures for affinity purification of in vitro transcribed RNA have been reported (Cheong, H. K., et al. (2004) supra; Kieft, J. S. and Batey, R. T. (2004) supra; Batey, R. T. and Kieft, J. S. (2007), supra; Pereira, M. J. et al. (2010), supra). They all incorporate four main steps: 1) transcription of a hybrid RNA that contains both the RNA of interest and a 3′-affinity tag; 2) immobilization of the transcribed RNA on an affinity matrix; 3) a wash step to remove impurities from the affinity matrix; and 4) elution of the target RNA by cleavage of the affinity tag. The RNA immobilization and cleavage steps are important aspects of the procedure. Several strategies have been employed for RNA immobilization, including RNA/DNA hybridization and high-affinity RNA/protein interactions (Cheong, H. K., et al. (2004) supra; Kieft, J. S. and Batey, R. T. (2004) supra; Batey, R. T. and Kieft, J. S. (2007), supra). RNA cleavage has been achieved in trans using a DNAzyme, however it requires additional purification steps to remove the co-eluting enzyme (Cheong, H. K., et al. (2004) supra). The use of RNA tags containing activatable ribozymes has been shown to substantially simplify the procedure, since no additional purification step, other than a buffer exchange, is required after the RNA elution (Kieft, J. S. and Batey, R. T. (2004) supra; Batey, R. T. and Kieft, J. S. (2007), supra); Boese, B. J. et al. (2008) Nucleosides Nucleotides & Nucleic Acids, 27, 949-966; Vicens, Q. et al. (2009), supra; Keel, A. Y. et al. (2009), supra). Recently, a method that exploits a His-tagged MS2 coat protein attached to a Ni-NTA resin for RNA immobilization and the glmS ribozyme activated by glucosamine-6-phosphate (Glc6NP) for RNA elution was described (Batey, R. T. and Kieft, J. S. (2007), supra). However, like other procedures reported so far for affinity purification of transcribed RNA, it was not developed to maximize RNA purity and yield. Thus, it is not clear from previous reports, if affinity purification methods can reliably produce RNA samples with the yields and purity levels required for the most demanding applications, such as accurate biochemical, biophysical and structural characterizations.

There is thus a need for the development of novel reagents and methods for the immobilization and purification of RNA that permits to achieve good yields and/or purity levels.

The present description refers to a number of documents, the content of which is herein incorporated by reference in their entirety.

### SUMMARY OF THE INVENTION

In a first aspect, the present invention provides a construct for immobilizing a bacteriophage boxB-comprising RNA on a solid support, said construct comprising:
a boxB RNA binding peptide;
a peptide linker linked to the C-terminus of said bacteriophage N peptide; and
an immobilizing moiety capable of binding to said solid support, wherein said immobilizing moiety is linked to said peptide linker.

In an embodiment, the above-mentioned boxB RNA binding peptide binds to said bacteriophage boxB with a dissociation constant (KD) of about 2×10⁻⁸ M or less. In a further embodiment, the above-mentioned boxB RNA binding peptide binds to said bacteriophage boxB with a dissociation constant (KD) of about 1×10⁻⁹ M or less.

In an embodiment, the above-mentioned boxB RNA binding peptide is a bacteriophage N peptide.

In a further embodiment, the above-mentioned bacteriophage N peptide comprises a domain of formula I (SEQ ID NO:1):

X¹—X²-A/S—X³—X⁴—R/K—X⁵—X⁶—X⁷—R/K—R/K—X⁸—X⁹—X¹⁰—X¹¹—X¹²—X¹³—X¹⁴ (I)

wherein
X¹ is any amino acid or is absent;
X² is A, D, T or N;
X³ is Q, R or K;
X⁴ is A, T or S;
X⁵ is Y or R
X⁶ is R, K or H;
X⁷ is E or A;
X⁸ is any amino acid;
X⁹ is any amino acid;
X¹⁰ is any amino acid;
X¹¹ is any amino acid;
X¹² is any amino acid;
X¹³ is any amino acid; and
X¹⁴ is any amino acid.

In a further embodiment, X¹ is M or G, X⁸ is A or R; X⁹ is E, K or M; X¹⁰ is K, L or E; X¹¹ is Q, I, A, or R; X¹² is A or I; X¹³ is Q, E or T; and/or X¹⁴ is W, R or L.

In yet a further embodiment, X¹ is G; X² is N; and/or X³ is K.

In a further embodiment, the above-mentioned domain is Met-Asp-Ala-Gln-Thr-Arg-Arg-Arg-Glu-Arg-Arg-Ala-Glu-Lys-Gln-Ala-Gln-Trp (MDAQTRRRERRAEKQAQW, SEQ ID NO:2); Gly-Asn-Ala-Lys-Thr-Arg-Arg-Arg-Glu-Arg-Arg-Ala-Glu-Lys-Gln-Ala-Gln-Trp (G NAKTRRRERRAEKQAQW, SEQ ID NO:3) or Gly-Asn-Ala-Lys-Thr-Arg-Arg-His-Glu-Arg-Arg-Arg-Lys-Leu-Ala-Ile-Glu-Arg (GNAKTRRHERRRKLAIER, SEQ ID NO:4).

In an embodiment, the above-mentioned peptide linker is a poly-glycine or poly-glycine/alanine linker.

In another embodiment, the above-mentioned peptide linker is a 20-residue peptide linker.

In a further embodiment, the above-mentioned peptide linker consists of the sequence (Gly-Ala)₁₀.

In an embodiment, the above-mentioned immobilizing moiety is a Glutathione S-transferase (GST) polypeptide.

In an embodiment, the above-mentioned solid support is a Glutathione Sepharose™ bead.

In an embodiment, the above-mentioned bacteriophage boxB-comprising RNA further comprises a target RNA which is targeted for immobilization.

In another aspect, the present invention provides a method for immobilizing a target RNA, said method comprising: (a) providing a bacteriophage boxB-comprising target RNA comprising a bacteriophage boxB RNA and the target RNA; and (b) contacting the bacteriophage boxB-comprising target RNA of (a) with the above-mentioned construct bound to a solid support.

In another aspect, the present invention provides a method for immobilizing a target RNA, said method comprising: (a) providing a bacteriophage boxB-comprising target RNA comprising a bacteriophage boxB RNA and the target RNA; (b) contacting the bacteriophage boxB-comprising target RNA of (a) with the above-mentioned construct, thereby to obtain a complex comprising the bacteriophage boxB-comprising target RNA bound to the construct; and (c) contacting the complex with a solid support comprising a ligand capable of binding to the immobilizing moiety.

In an embodiment, the above-mentioned method further comprises preparing the bacteriophage boxB-comprising target RNA by incorporating a bacteriophage boxB sequence to said target RNA.

In another aspect, the present invention provides a method for purifying a target RNA, said method comprising: (a) providing an affinity tag-comprising target RNA comprising an affinity tag and the target RNA, wherein said affinity tag comprises a bacteriophage boxB sequence and an activatable ribozyme sequence; (b) contacting the affinity tag-comprising target RNA of (a) with the above-mentioned construct bound to a solid support; (c) inducing activation of said activatable ribozyme; and (d) collecting said target RNA.

In another aspect, the present invention provides a method for purifying a target RNA, said method comprising: providing an affinity tag-comprising target RNA comprising an affinity tag and the target RNA, wherein said affinity tag comprises a bacteriophage boxB sequence and an activatable ribozyme sequence; (b) contacting the affinity tag-comprising target RNA of (a) with the above-mentioned construct thereby to obtain a complex comprising the affinity tag-comprising target RNA bound to the construct; (c) contacting the complex with a solid support comprising a ligand capable of binding to the immobilizing moiety; (d) inducing activation of said activatable ribozyme; and (e) collecting said target RNA.

In an embodiment, the above-mentioned method further comprises preparing the affinity tag-comprising target RNA by incorporating the affinity tag to said target RNA.

In an embodiment, the above-mentioned inducing activation of said activatable ribozyme comprises contacting the solid support with an agent capable of activating said activatable ribozyme.

In an embodiment, the above-mentioned bacteriophage boxB sequence is a bacteriophage lambda boxB sequence.

In an embodiment, the above-mentioned bacteriophage boxB sequence is incorporated at the 3′ end of said target RNA.

In an embodiment, the above-mentioned method further comprises incorporating a linker at the 3′ end of said target RNA. In a further embodiment, the above-mentioned 3′ linker is a 1- or 2-nucleotide linker. In a further embodiment, the above-mentioned 3′ linker is GA, GG, GC, GU or A.

In an embodiment, the above-mentioned bacteriophage boxB sequence is incorporated into the variable apical P1 stem-loop of said activatable ribozyme sequence.

In another embodiment, the above-mentioned activatable ribozyme sequence is a Glucosamine-6-phosphate activated ribozyme (glmS ribozyme) sequence. In a further embodiment, the above-mentioned glmS ribozyme sequence is a Bacillus anthracis glmS ribozyme sequence.

In an embodiment, the above-mentioned agent capable of activating said activatable ribozyme is Glucosamine-6-phosphate (Glc6NP).

In an embodiment, the above-mentioned method further comprises a step of contacting the solid support with a saline solution to disrupt binding of said affinity tag with said N peptide, subsequent to the step of collecting said target RNA. In a further embodiment, the above-mentioned saline solution is a 2.5M sodium chloride (NaCl) solution.

In an embodiment, the above-mentioned immobilizing moiety is a Glutathione S-transferase (GST) polypeptide and said solid support is a Glutathione Sepharose™ bead, and wherein said method further comprises (e) contacting the solid support with a glutathione (GSH) solution to disrupt binding of said construct with said solid support.

In an embodiment, the above-mentioned method further comprises a washing step subsequent to said contacting with a solid support and prior to said inducing activation of said activatable ribozyme.

In another aspect, the present invention provides a kit for immobilizing a target RNA, said kit comprising the above-mentioned construct and instructions for immobilizing the target RNA using the above-mentioned method.

In another aspect, the present invention provides a kit for immobilizing a target RNA, said kit comprising the above-mentioned construct and a nucleic acid construct comprising a sequence encoding a bacteriophage boxB RNA.

In an embodiment, the above-mentioned kit further comprises instructions for immobilizing the target RNA using the above-mentioned method.

In another embodiment, the above-mentioned method further comprises a solid support comprising a ligand capable of binding to the immobilizing moiety.

In another aspect, the present invention provides a kit for purifying a target RNA, said kit comprising the above-mentioned construct and instructions for purifying the target RNA using the above-mentioned method.

In another aspect, the present invention provides a kit for purifying a target RNA, said kit comprising the above-mentioned construct and a nucleic acid construct comprising a first sequence encoding a bacteriophage boxB RNA and a second sequence encoding an activatable ribozyme.

In another aspect, the present invention provides a kit for purifying a target RNA, said kit comprising the above-mentioned construct and a first nucleic acid construct comprising a first sequence encoding a bacteriophage boxB RNA and a second nucleic acid construct comprising a second sequence encoding an activatable ribozyme.

In an embodiment, the above-mentioned kit further comprises instructions for purifying the target RNA using the above-mentioned method.

In another embodiment, the above-mentioned kit further comprises a solid support comprising a ligand capable of binding to the immobilizing moiety.

In another embodiment, the above-mentioned kit further comprises an agent capable of activating said activatable ribozyme.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the general strategy for affinity batch purification of a desired RNA based on the λboxB RNA/λN-peptide interaction. In this schematic, the RNA is fused to an “ARiBo” tag (Activatable Ribozyme with BoxB RNA) and purified via binding to an λN peptide fused to a GST protein, for immobilisation on GSH-Sepharose™ beads. RNA elution is triggered by addition of GlcN6P, which activates the glmS ribozyme of the ARiBo tag. The affinity matrix can be regenerated by stepwise incubation with 2.5 M NaCl and 20 mM GSH, as described in Example 1;

FIG. 2A shows the primary and secondary structures of one of the RNA of interest used in the studies described herein, the U65C mutant of the B. subtilis pbuE adenine riboswitch aptamer (SEQ ID NO:5);

FIG. 2B shows the primary and secondary structures of three ARiBo tags tested in the studies described herein (Example 5). The ARiBo tags contain the B. anthracis glmS sequence except for P1 substitutions and 3′ extensions with λboxB RNA sequence or U1A binding site, as shown;

FIG. 2C shows the nucleotide sequences of the ARiBo1, ARiBo2 and ARiBo3 tags of FIG. 2B. The regions corresponding to the λboxB RNA sequences are underlined;

FIG. 2D shows the nucleotide sequences of other representative ARiBo tags that may be used for RNA immobilization. The regions corresponding to the P22boxB RNA (in ARiBo4 and ARiBo5) and λboxB RNA (in ARiBo6 and ARiBo7) sequences are underlined;

FIG. 3 shows the nomenclature and schematic diagram of the different GST/λN fusion proteins tested in the studies described herein. The λN peptide contains the first 22 residues of the λN protein and the λN⁺ peptide is a G1N2K4 triple mutant of the λN peptide (Austin, R. J., et al. (2002) J Am Chem Soc, 124, 10966-10967).

FIGS. 4A and 4B show typical small-scale affinity batch purifications of RSA_{U65C} analyzed on a SYBR™ Gold stained denaturing polyacrylamide gel. The RSA_{U65C} was synthesized as an ARiBo1-fused RNA. The GST/λN fusion protein used was either GST-λN (FIG. 4A) or λN⁺-L⁺-GST (FIG. 4B). Aliquots from each purification step were loaded on the gel (LS: loading supernatant; W1-3: washes; E1-3: elutions; and NaCl: matrix regeneration with 2.5 M NaCl) in amounts shown, where 1× correspond to 50 ng of ARiBo-fused RSA_{U65C} present in the transcription reaction or 16 ng of RSA_{U65C} to be purified. In addition, standard amounts of ARiBo-fused RSA_{U65C} from the transcription reaction, purified RSA_{U65C}, and RSA_{U65C} cleaved in the transcription reaction were loaded for quantitative analysis of the purification (see Example 1). Bands corresponding to the ARiBo-fused RSA_{U65C}, the ARiBo1 tag and the desired RNA (RSA_{U65C}) are indicated on the right of the gel;

FIG. 5 shows the effect of sequence at the 3′-end of the desired RNA for cleavage by the glmS ribozyme of the ARiBo1 tag. ARiBo1-fused RNAs with the original RSA_{U65C} sequence (AG linker; FIG. 2A) or carrying mutations at the 3′-end (GG, GU, GC and A linkers) were cleaved under different conditions. All cleavage reactions were performed at 37° C. in solution containing a 1/50 dilution of the transcription reaction, 10 mM MgCl₂, 20 mM Tris buffer pH 7.6, but with different concentrations of GlcN6P and for different amounts of time, as indicated above each lane. The mobility of the RNA precursor and products is marked with arrows on the right side of the gel. The percentage of cleavage for each condition is given below the gel;

FIGS. 6A and 6B shows imino regions of the 1D ¹H NMR spectra of 0.35 mM RSA_{U65C} purified by affinity batch purification (FIG. 6A) and a standard purification protocol based on denaturing gel electrophoresis (FIG. 6B);

FIG. 7A shows the amino acid sequences of the two bacteriophage N peptides used in the studies described herein (λN: SEQ ID NO: 6, λN⁺: SEQ ID NO: 7);

FIG. 7B shows a schematic representation of the protein expression vectors used in the studies described herein. For the three pet42a-derived vectors that express a λN⁺/GST fusion protein, the fusion protein is synthesized with an initiator methionine, which is absent in the purified protein;

FIG. 8A shows the primary structure of the cloning region within the RNA expression vector pRSAU65C-ARiBo1 (SEQ ID NO:8). The RNA of interest is in grey, the ARiBo1 tag in italic, and the λboxB sequence within the ARiBo1 is in bold. The relevant restriction sites and the T7 promoter are labeled. The arrowhead indicates the cleavage site of the glmS ribozyme;

FIG. 8B shows a schematic representation of the RNA expression vectors used in the studies described herein. The relevant restriction sites are indicated;

FIG. 9A shows a description of λN⁺-L⁺-GST, a fusion protein containing the G1N2K4 mutant of the bacteriophage N₁₋₂₂ peptide (λN⁺, SEQ ID NO:7) and a (Gly-Ala)₁₀ linker (L⁺) at the N terminus of GST;

FIGS. 9B and 9C show coomassie-stained SDS polyacrylamide gels of fractions collected at various stages of purification. In FIG. 9B, lane 1: Molecular weight marker; lanes 2 and 3: pre-induction (lane 2) and post-induction (lane 3) whole cell extract; lane 4: soluble E. coli lysate following ultracentrifugation; lanes 5-7: glutathione elutions 1-3 from the GSH-Sepharose™; lanes 8 and 9: proteins still present in the supernatant (lane 8) and the resin (lane 9) following elution with glutathione. In FIG. 9C lane 1: Molecular weight marker; lanes 2-14: fractions 41-54 from the SP-Sepharose™ column. Fractions 44 to 51 (lanes 5 to 12), inclusively, were selected for dialysis and storage;

FIGS. 10A and 10B show quality controls of the purified λN⁺-L⁺-GST fusion protein. FIG. 10A: purity and stability of the purified protein. Lane 1: molecular weight marker; lanes 2-7: 0.25, 0.5, 1.0, 2.0, 5.0 and 10 μg of purified λN⁺-L⁺-GST; lanes 8-10: 5 μg of purified λN⁺-L⁺-GST following incubations for 1, 2 and 4 h at 37° C. (b) RNase contamination assay. Lane 1: molecular weight marker with RNA size given in terms of the number of nucleotides (nts); lanes 2-5: 50 ng of TL-let-7g RNA following incubations for 0, 1, 2 and 4 h at 37° C.; lanes 6-9: 50 ng of TL-let-7g RNA following incubations for 0, 1, 2 and 4 h at 37° C. in the presence of purified λN⁺-L⁺-GST; lanes 10-13: 2.5, 10, 25 and 50 ng of gel-purified TL-let-7g RNA;

FIG. 11A shows the structure/sequence of the RNA of interest used in Example 8, the terminal loop of the let-7g precursor miRNA from Mus musculus (TL-let-7g, SEQ ID NO: 9). FIG. 11B shows the structure/sequence of the pARiBo1 plasmid used for cloning and transcription (SEQ ID NO:10). The restriction sites (HindIII, ApaI and EcoRI) are boxed and the T7 promoter is indicated;

FIG. 12 shows the GlmS ribozyme cleavage optimization of the ARiBo-fusion RNA. All cleavage reactions were performed at 37° C. in 100-4 solution containing 3 μL of the standard transcription reaction, 10 mM MgCl₂, 20 mM Tris buffer pH 7.6, but with different concentrations of GlcN6P and for different amounts of time (in min), as indicated above each lane. The mobility of the RNA precursor and products is marked with arrows on the left side of the gel. The percentage of cleavage for each condition is given below the gel. Standard amounts of purified TL-let-7g were loaded for quantitative analysis of the transcription;

FIGS. 13A and 13B show the affinity batch purification of the TL-let-7g RNA. FIG. 13A: General purification scheme. The TL-let-7g RNA is synthesized as an ARiBo1-fusion RNA (TL-let-7g-ARiBo1) and immobilized on GSH-Sepharose™ beads via a λN⁺-L⁺-GST fusion protein. RNA elution is triggered by addition of GlcN6P, which activates the glmS ribozyme of the ARiBo tag. The resin matrix is partly regenerated by incubation with 2.5 M NaCl. FIG. 13B: Small-scale affinity purification of TL-let-7g RNA analyzed on a 10% denaturing polyacrylamide gel stained with SYBR™ Gold. Aliquots from each purification steps were loaded on the gel (LE: loading eluate; W1-3: washes; E1-3: RNA elutions; and NaCl: matrix regeneration with 2.5 M NaCl) in amounts shown, where 1× correspond to 50 ng of TL-let-7g-ARiBo1 present in the transcription reaction or 11.9 ng of TL-let-7g to be purified. In addition, standard amounts of the TL-let-7g-ARiBo1 fusion RNA from the transcription reaction, purified TL-let-7g and TL-let-7g after cleavage of the fusion RNA in the transcription reaction were loaded for quantitative analysis of the purification. Bands corresponding to the TL-let-7g-ARiBo1 fusion RNA, the ARiBo1 tag and the desired RNA (TL-let-7g) are indicated on the right of the gel.

### DISCLOSURE OF INVENTION

In the studies described herein, a matrix has been developed that permits immobilization and purification of an RNA of interest with high yield and purity. The matrix is based on the high affinity between a bacteriophage boxB RNA and phage-derived peptides, such as bacteriophage N peptides.

Accordingly, the present invention provides a peptide/polypeptide construct for immobilizing a bacteriophage boxB-comprising RNA on a solid support/matrix, said construct comprising:
a boxB RNA binding peptide;
a moiety capable of binding to the solid support (an immobilizing moiety); and
a peptide linker located between the boxB RNA binding peptide and the moiety.

The above-mentioned immobilizing moiety may be N- or C-terminal relative to the boxB RNA binding peptide. In an embodiment, the above-mentioned immobilizing moiety is C-terminal relative to the boxB RNA binding peptide.

In another aspect, the present invention provides a peptide/polypeptide construct for immobilizing a bacteriophage boxB-comprising RNA on a solid support/matrix, said construct comprising:
a boxB RNA binding peptide;
a peptide linker linked/attached to the C-terminal of said boxB RNA binding peptide; and
a moiety capable of binding to said solid support, wherein said moiety is linked to said peptide linker.

The term “bacteriophage boxB RNA” refers to a RNA hairpin sequence comprising short stems (typically 5-7 bp) and 5- or 6-nucleotide loop found in some bacteriophages, notably the Lambda family of bacteriophages (e.g., λ, φ21, and P22). Bacteriophage boxB RNAs have a strong affinity for specific phage-derived polypeptides/peptides involved in the regulation of transcription (e.g., transcription termination, transcription anti-termination), such as bacteriophage N proteins and Coliphage HK022 nun protein, and more particularly to the amino-terminal region of such polypeptides/peptides. Examples of bacteriophage boxB RNA include RNAs comprising the following sequences (Cilley and Williamson, RNA (2003) 9: 663-676; Neely and Friedman, Molecular Microbiology (2000) 38(5): 1074-1085; Chattopadhyay, S. et al. (1995) Proc. Natl. Acad. Sci. 92; 4061-4065):

| (bacteriophage λ, SEQ ID NO: 11) | |
|---|---|
| 1) | GCCCU GAAAA AGGGC; |
| (bacteriophage λ, SEQ ID NO: 12) | |
| 2) | GCCCU GAAGA AGGGC; |
| (bacteriophage φ21, SEQ ID NO: 13) | |
| 3) | UUCACCU CUAACC GGGUGAG; |
| (bacteriophage φ21, SEQ ID NO: 14) | |
| 4) | UCUCAAC CUAACC GUUGAGA; |
| (bacteriophage P22, SEQ ID NO: 15) | |
| 5) | ACCGCC CACAA CGCGGU; |
| (bacteriophage P22, SEQ ID NO: 16) | |
| 6) | UGCGCU GACAA AGCGCG; |
| (bacteriophage H-19, SEQ ID NO: 17) | |
| 7) | UCGCU GACAA AGCGA; |
| (bacteriophage H-19, SEQ ID NO: 18) | |
| 8) | GCGCU GACAA AGCGC; |
| (bacteriophage HK97, SEQ ID NO: 19) | |
| 9) | UCGCU GACAA AGCGA; |
| (bacteriophage HK97, SEQ ID NO: 20) | |
| 10) | GCGGU CACAA AGCGC; |
| (bacteriophage 933W, SEQ ID NO: 21) | |
| 11) | UCGCU GACAA AGCGA; |
| (bacteriophage 933W, SEQ ID NO: 22) | |
| 12) | GCGCU GACAA AGCGC; |
| (bacteriophage λ, SEQ ID NO: 23) | |
| 13) | GCCUG AAAAA GGGC; |
| (bacteriophage λ, SEQ ID NO: 24) | |
| 14) | GCCUG GAAAA GGGC; |
| (bacteriophage λ, SEQ ID NO: 25) | |
| 15) | GCCUG UAAAA GGGC; |
| (bacteriophage λ, SEQ ID NO: 26) | |
| 16) | GCCUG CAAAA GGGC; |
| (bacteriophage λ, SEQ ID NO: 27) | |
| 17) | GCCUG AGAAA GGGC; |
| (bacteriophage λ, SEQ ID NO: 28) | |
| 18) | GCCUG AAGAA GGGC; |
| (bacteriophage λ, SEQ ID NO: 29) | |
| 19) | GCCUG AAUAA GGGC; |
| (bacteriophage λ, SEQ ID NO: 30) | |
| 20) | GCCUG AACAA GGGC; |
| (bacteriophage λ, SEQ ID NO: 31) | |
| 21) | GCCUG AAAGA GGGC; |
| (bacteriophage λ, SEQ ID NO: 32) | |
| 22) | GCCUG AAAUA GGGC; |
| and | |
| (bacteriophage λ, SEQ ID NO: 33) | |
| 23) | GCCUG AAACA GGGC. |

In an embodiment, the above-mentioned bacteriophage boxB RNA is a bacteriophage lambda (λ) boxB RNA sequence. In a further embodiment, the above-mentioned bacteriophage boxB RNA comprises the following sequence: GCCCU GAAGA AGGGC (SEQ ID NO:12). In a further embodiment, the above-mentioned bacteriophage boxB RNA comprises the following sequence: GGCCCU GAAGA AGGGCU (SEQ ID NO: 34).

In another embodiment, the above-mentioned bacteriophage boxB RNA comprises the consensus sequence GNNRA or GNRNN, wherein N=A, G, C or U and R=A or G.

As used herein, boxB RNA binding peptide refers to phage-derived peptides capable of binding with high affinity to a boxB RNA sequence. Typically, these phage-derived peptides are derived from specific regions of proteins (generally arginine-rich motif located in the N-terminal portion) involved in the regulation of transcription (e.g., termination), such as bacteriophage N proteins and coliphage HK022 Nun proteins.

As used herein, bacteriophage N peptide refers to an arginine-rich peptide motifs (ARMs) derived from bacteriophage N proteins and having affinity for a bacteriophage boxB RNA. Examples of bacteriophage N peptides include peptides comprising the following sequences (Cilley and Williamson, RNA (2003) 9: 663-676; Austin et al., 2002, supra), with the residues conserved between the different sequences underlined and the residues mutated in bold:

(bacteriophage λ, SEQ ID NO: 2)
1) MDAQTRRRER RAEKQAQW;
(bacteriophage φ21, SEQ ID NO: 35)
2) GTAKSRYKAR RAELIAER;
(bacteriophage P22, SEQ ID NO: 36)
3) GNAKTRRHER RRKLAIER;

Mutated bacteriophage N peptides are described, for example, in Austin et al. (Austin et al., 2002, supra), and include mutated bacteriophage A N peptides exhibiting increased affinity for bacteriophage boxB RNA and comprising the following sequences (residues mutated in bold);

(SEQ ID NO: 37)
1) MNAQTRRRER R AEKQAQWKAAN;
(SEQ ID NO: 38)
2) MNAKTRRRER R AEKQAQWKAAN;
(SEQ ID NO: 39)
3) MNARTRRRER R AEKQAQWKAAN;
(SEQ ID NO: 7)
4) GNAKTRRRER R AEKQAQWKAAN;
(SEQ ID NO: 40)
5) GNARTRRRER R AEKQAQWKAAN;
(SEQ ID NO: 41)
6) GNARTRRRER R AMERATLPQVL.

Other bacteriophage N peptide mutants are disclosed in Su et al., Biochemistry (1997), 36: 12722-12732:

(DAQTRRRERRAEKQAQWKAAN, SEQ ID NO: 42)

| | |
|---|---|
| 1) | Asp-Ala-Gln-Thr-Arg-Arg-Arg-Glu-Arg-Arg-Ala- |
| | Glu-Lys-Gln-Ala-Gln-Trp-Lys-Ala-Ala-Asn |

(AAQTRRRERRAEKQAQWKAAN, SEQ ID NO: 43)

| | |
|---|---|
| 2) | Ala-Ala-Gln-Thr-Arg-Arg-Arg-Glu-Arg-Arg-Ala- |
| | Glu-Lys-Gln-Ala-Gln-Trp-Lys- Ala-Ala-Asn |

(DSQTRRRERRAEKQAQWKAAN, SEQ ID NO: 44)

| | |
|---|---|
| 3) | Asp-Ser-Gln-Thr-Arg-Arg-Arg-Glu-Arg-Arg-Ala- |
| | Glu-Lys-Gln-Ala-Gln-Trp-Lys-Ala-Ala-Asn |

(DAQARRRERRAEKQAQWKAAN, SEQ ID NO: 45)

| | |
|---|---|
| 4) | Asp-Ala-Gln-Ala-Arg-Arg-Arg-Glu-Arg-Arg-Ala- |
| | Glu-Lys-Gln-Ala-Gln-Trp-Lys- Ala-Ala-Asn |

(DAQTKRRERRAEKQAQWKAAN, SEQ ID NO: 46)

| | |
|---|---|
| 5) | Asp-Ala-Gln-Thr-Lys-Arg-Arg-Glu-Arg-Arg-Ala- |
| | Glu-Lys-Gln-Ala-Gln-Trp-Lys-Ala-Ala-Asn |

(DAQTRRRARRAEKQAQWKAAN, SEQ ID NO: 47)

| | |
|---|---|
| 6) | Asp-Ala-Gln-Thr-Arg-Arg-Arg-Ala-Arg-Arg-Ala- |
| | Glu-Lys-Gln-Ala-Gln-Trp-Lys-Ala-Ala-Asn |

(DAQTRRREKRAEKQAQWKAAN, SEQ ID NO: 48)

| | |
|---|---|
| 7) | Asp-Ala-Gln-Thr-Arg-Arg-Arg-Glu-Lys-Arg-Ala- |
| | Glu-Lys-Gln-Ala-Gln-Trp-Lys-Ala-Ala-Asn |

(DAQTRRRERKAEKQAQWKAAN, SEQ ID NO: 49)

| | |
|---|---|
| 8) | Asp-Ala-Gln-Thr-Arg-Arg-Arg-Glu-Arg-Lys-Ala- |
| | Glu-Lys-Gln-Ala-Gln-Trp-Lys-Ala-Ala-Asn |

(DAQTRRRERRGEKQAQWKAAN, SEQ ID NO: 50)

| | |
|---|---|
| 9) | Asp-Ala-Gln-Thr-Arg-Arg-Arg-Glu-Arg-Arg-Gly- |
| | Glu-Lys-Gln-Ala-Gln-Trp-Lys-Ala-Ala-Asn |

(DAQTRRRERRAAKQAQWKAAN, SEQ ID NO: 51)

| | |
|---|---|
| 10) | Asp-Ala-Gln-Thr-Arg-Arg-Arg-Glu-Arg-Arg-Ala- |
| | Ala-Lys-Gln-Ala-Gln-Trp-Lys-Ala-Ala-Asn |

(DAQTRRRERRAPKQAQWKAAN, SEQ ID NO: 52)

| | |
|---|---|
| 11) | Asp-Ala-Gln-Thr-Arg-Arg-Arg-Glu-Arg-Arg-Ala- |
| | Pro-Lys-Gln-Ala-Gln-Trp-Lys-Ala-Ala-Asn |

(DAQTRRRERRAERQAQWKAAN, SEQ ID NO: 53)

| | |
|---|---|
| 12) | Asp-Ala-Gln-Thr-Arg-Arg-Arg-Glu-Arg-Arg-Ala- |
| | Glu-Arg-Gln-Ala-Gln-Trp-Lys-Ala-Ala-Asn |

(DAQTRRRERRAEKQGQWKAAN, SEQ ID NO: 54)

| | |
|---|---|
| 13) | Asp-Ala-Gln-Thr-Arg-Arg-Arg-Glu-Arg-Arg-Ala- |
| | Glu-Lys-Gln-Gly-Gln-Trp-Lys-Ala-Ala-Asn |

(DAQTRRRERRAEKQAAWKAAN, SEQ ID NO: 55)

| | |
|---|---|
| 14) | Asp-Ala-Gln-Thr-Arg-Arg-Arg-Glu-Arg-Arg-Ala- |
| | Glu-Lys-Gln-Ala-Ala-Trp-Lys-Ala-Ala-Asn |

(DAQTRRRERRAEKQAQFKAAN, SEQ ID NO: 56)

| | |
|---|---|
| 15) | Asp-Ala-Gln-Thr-Arg-Arg-Arg-Glu-Arg-Arg-Ala- |
| | Glu-Lys-Gln-Ala-Gln-Phe-Lys-Ala-Ala-Asn |

(DAQTRRRERRAEKQAQYKAAN, SEQ ID NO: 57)

| | |
|---|---|
| 16) | Asp-Ala-Gln-Thr-Arg-Arg-Arg-Glu-Arg-Arg-Ala- |
| | Glu-Lys-Gln-Ala-Gln-Tyr-Lys-Ala-Ala-Asn |

(DAQTRRRERRAEKQAQWAAAN, SEQ ID NO: 58)

| | |
|---|---|
| 17) | Asp-Ala-Gln-Thr-Arg-Arg-Arg-Glu-Arg-Arg-Ala- |
| | Glu-Lys-Gln-Ala-Gln-Trp-Ala-Ala-Ala-Asn |

(DAQTRRRERRAEKQAQWKGAN, SEQ ID NO: 59)

| | |
|---|---|
| 18) | Asp-Ala-Gln-Thr-Arg-Arg-Arg-Glu-Arg-Arg-Ala- |
| | Glu-Lys-Gln-Ala-Gln-Trp-Lys-Gly-Ala-Asn |

(DAQTRRRERRAEKQAQWKAGN, SEQ ID NO: 60)

| | |
|---|---|
| 19) | Asp-Ala-Gln-Thr-Arg-Arg-Arg-Glu-Arg-Arg-Ala- |
| | Glu-Lys-Gln-Ala-Gln-Trp-Lys-Ala-Gly-Asn |

(DAQTRRRERRAEKQAQWKAAA, SEQ ID NO: 61)

| | |
|---|---|
| 20) | Asp-Ala-Gln-Thr-Arg-Arg-Arg-Glu-Arg-Arg-Ala- |
| | Glu-Lys-Gln-Ala-Gln-Trp-Lys-Ala-Ala-Ala |

Accordingly, in an embodiment, the above-mentioned bacteriophage N peptide comprises a domain of formula I (SEQ ID NO:1):

X¹—X²-A/S—X³—X⁴—R/K—X⁵—X⁶—X⁷—R/K—R/K—X⁸—X⁹—X¹⁰—X¹¹—X¹²—X¹³—X¹⁴ (I)

wherein

X¹ is any amino acid or is absent; X² is A, D, T or N; X³ is Q, R or K; X⁴ is A, T or S; X⁵ is Y or R; X⁶ is R, K or H; X⁷ is E or A; X⁸ is any amino acid; X⁹ is any amino acid; X¹⁰ is any amino acid; X¹¹ is any amino acid; X¹² is any amino acid; X¹³ is any amino acid; and X¹⁴ is any amino acid.

In embodiments, A/S at position 3 is A, R/K at position 6 is R, R/K at position 10 is R and/or R/K at position 11 is R.

In another embodiment, the above-mentioned bacteriophage N peptide comprises a domain of formula II (SEQ ID NO:62):

X¹—X²-A—X³—X⁴—R—X⁵—X⁶—X⁷—R—R—X⁸—X⁹—X¹⁰—X¹¹—X¹²—X¹³—X¹⁴ (II)

wherein

X¹ is any amino acid or is absent; X² is A, D, T or N; X³ is Q, R or K; X⁴ is A, T or S; X⁵ is Y or R; X⁶ is R, K or H; X⁷ is E or A; X⁸ is any amino acid; X⁹ is any amino acid; X¹⁰ is any amino acid; X¹¹ is any amino acid; X¹² is any amino acid; X¹³ is any amino acid; and X¹⁴ is any amino acid.

In embodiments:

X¹ is M or G; X⁸ is A or R; X⁹ is E, K or M; X¹⁰ is K, L or E; X¹¹ is Q, I, A, or R; X¹² is A or I; X¹³ is Q, E or T; and/or X¹⁴ is W, R or L.

In an embodiment, X¹ is Gly; X² is Asn; and/or X³ is Lys.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of peptides. Examples of naturally-occurring amino acids are glycine, alanine, valine, leucine, isoleucine, serine, threonine, etc. Other amino acids include for example norleucine, norvaline, cyclohexyl alanine, biphenyl alanine, homophenyl alanine, naphthyl alanine, pyridyl alanine, phenyl alanines substituted at the ortho, para and meta positions with alkoxy, halogen or nitro groups etc. These amino acids are well known in the art of biochemistry/peptide chemistry.

Therefore, the above-mentioned polypeptide construct (or any part thereof, e.g., the linker, the immobilizing moiety, and/or the boxB RNA binding peptide) may comprise L-amino acids, D-amino acids or a combination/mixture thereof. In an embodiment, the above-mentioned polypeptide construct (or any part thereof, e.g., the linker, the immobilizing moiety, and/or the boxB RNA binding peptide) comprises only L-amino acids.

In another embodiment, the above-mentioned domain is Met-Asp-Ala-Gln-Thr-Arg-Arg-Arg-Glu-Arg-Arg-Ala-Glu-Lys-Gln-Ala-Gln-Trp (MDAQTRRRERRAEKQAQW, SEQ ID NO:2); Gly-Asn-Ala-Lys-Thr-Arg-Arg-Arg-Glu-Arg-Arg-Ala-Glu-Lys-Gln-Ala-Gln-Trp (GNAKTRRRERRAEKQAQW, SEQ ID NO:3) or Gly-Asn-Ala-Lys-Thr-Arg-Arg-His-Glu-Arg-Arg-Arg-Lys-Leu-Ala-Ile-Glu-Arg (GNAKTRRHERRRKLAIER, SEQ ID NO:4). In a further embodiment, the above-mentioned bacteriophage N peptide is Gly-Asn-Ala-Lys-Thr-Arg-Arg-Arg-Glu-Arg-Arg-Ala-Glu-Lys-Gln-Ala-Gln-Trp (GNAKTRRRERRAEKQAQW, SEQ ID NO:3).

Bacteriophage N peptide as used herein also encompass derivatives of naturally occurring or mutated N peptides, for example acridine derivatives as described in Qi et al., Biochemistry (2010) 49: 5782-5789.

In an embodiment, the above-mentioned boxB RNA binding peptide is a peptide derived from Coliphage HK022 Nun protein, and more particularly comprises within residues 1 to 44 (e.g., residues 10-44 or 20-44) of the Coliphage HK022 Nun protein (Faber et al., J. Biol. Chem. 276(34): 32064-32070). The sequence corresponding to residues 10-44 is as follows: DSGQNRKVSDRGLTSRDRRRIARWEKRIAYALKNG (SEQ ID NO:63).

In an embodiment, the above-mentioned boxB RNA binding peptide (e.g., bacteriophage N peptide) binds to said bacteriophage boxB with a dissociation constant (K_{D}) of about 2×10⁻⁸ M or less at physiological salt concentrations (about 150 mM).

In further embodiments, the above-mentioned boxB RNA binding peptide (e.g., bacteriophage N peptide) binds to said bacteriophage boxB with a K_{D} of about 5×10⁻⁸ M or less, about 2×10⁻⁸ M or less, 1×10⁻⁸ M or less, about 5×10⁻⁹ M or less, about 1×10⁻⁹ M or less, or about 1×10⁻¹⁰ or less at physiological salt concentrations (about 150 mM).

In an embodiment, the above-mentioned boxB RNA binding peptide comprises from about 10 to about 40 amino acids, in further embodiments from about 10 to about 30, from about 13 to about 25, from about 15 to about 21 amino acids, from about 17 to about 20 amino acids.

In embodiments, the above-mentioned peptide/polypeptide construct comprises a plurality of boxB RNA binding peptide (e.g., bacteriophage N peptide) (either multiple copies of the same peptide, or different peptides). In a further embodiment, the above-mentioned construct comprises two boxB RNA binding peptide (e.g., two bacteriophage N peptides or one bacteriophage N peptide and one Coliphage HK022 Nun peptide). The plurality of boxB RNA binding peptides may be covalently linked either directly (e.g., through a peptide bond) or via a suitable linker moiety, e.g., a linker of one or more amino acids (e.g., a polyglycine linker or the like) or another type of chemical linker (e.g., a carbohydrate linker, a lipid linker, a fatty acid linker, a polyether linker, PEG, etc. (see, e.g., Hermanson (1996) Bioconjugate techniques).

In an embodiment, the above-mentioned boxB RNA binding peptide (e.g., bacteriophage N peptide) comprises at its carboxy-terminal end a peptide linker that link the bacteriophage N peptide and the moiety capable of binding to the solid support (immobilizing or binding moiety). Therefore, in an embodiment, the configuration of the peptide/polypeptide construct (from N— to C-terminal) is as follows:
boxB RNA binding peptide-peptide linker-immobilizing moiety

The peptide linker may be any amino acid sequence, such as a natural (e.g., a naturally occurring peptide or polypeptide) or an artificial (e.g., a synthetic, non-naturally occurring peptide or polypeptide) sequence, that permits the binding of the boxB RNA binding peptide to the boxB RNA sequence (e.g., that does not significantly interfere with the binding of the boxB RNA binding peptide to the boxB RNA sequence) and that permits the binding of the immobilizing moiety to the solid support (e.g., that does not significantly interfere with the binding of the immobilizing moiety to the solid support). In an embodiment, the above-mentioned peptide linker has a length of 1000 amino acids or less, for example between about 2, 3, 4 or 5 to about 1000, 900, 800, 700, 600 or 500 amino acids. In further embodiments, the above-mentioned peptide linker has a length of between about 2 to about 250, between about 2 to about 100, between about 2 to about 50, between about 4 and about 40, between about 6 to about 30, between about 8 to about 25, between about 10, 11, 12, 13, 14, 15 or 16, to about 24, 23, 22, 21 or 20 amino acids.

In an embodiment, the above-mentioned peptide linker is a poly-glycine, poly-alanine or a mixed poly-glycine/alanine linker, in a further embodiment a mixed, alternate poly-glycine/alanine linker. In another embodiment, the above-mentioned peptide linker is a 20-residue peptide linker. In a further embodiment, the above-mentioned peptide linker comprises the amino acid sequence (G-A)₁₀. In a further embodiment, the above-mentioned peptide linker consists of the amino acid sequence (G-A)₁₀.

As noted above, the above-mentioned construct further comprises a moiety capable of binding to said solid support (an immobilizing or binding moiety). The immobilizing moiety is linked to the peptide linker and is thus indirectly fused (i.e. through the peptide linker) to the C-terminal of the boxB RNA binding peptide (e.g., bacteriophage N peptide). The immobilizing moiety may be any moiety capable of binding, either directly or indirectly, to a solid support. The term “solid support” (or “solid matrix”) generally refers to is any material to which a biospecific ligand is covalently attached, such as a chromatrographic media (e.g., resin, gel, beads) that are generally used to purify separate molecules and macromolecules based on various properties (e.g., size, charge, affinity for a given ligand, etc.). Solid supports are well known in the art and include, for example, matrices of polyacrylamide resins or cross-linked polysaccharides such as cross-linked dextran (e.g., Sephadex™), cross-linked agarose (e.g., Sepharose™) and the like. In an embodiment, the solid support comprises a moiety or ligand capable of binding to the immobilizing moiety of the polypeptide construct.

Moieties capable of binding to a solid support useful for affinity purification are well known in the art. For example, polyhistidine tag (commonly referred to as His-tag) are moiety comprising a plurality of histidine residues (at least 5, typically 6) which are capable of binding a solid support that contains bound metal ions, and more particularly nickel or cobalt. Such solid supports are well known in the art and commercially available under the trade names Ni Sepharose™, NTA-agarose™, His60 Ni Superflow™, His Pur™ resin, or Talon™ resin. Other known binding moieties useful for affinity purification include biotin-based tag (which binds to Avidin, Streptavidin or analogs/derivatives thereof), Strep tag (a short peptide of 8 amino acids: WSHPQFEK) which binds to Strep-Tactin™ an engineered form of streptavidin (commercially available from Qiagen), as well as Glutathione S-transferase (GST) tag, which binds to a Gutathione™-containing solid support such as Glutathione Sepharose™ resin (GE Healthcare), ProCatch™ Glutathione Resin (Miltenyi Biotec) and Glutathione Superflow™ resin (Qiagen). Any affinity tag-based system may be used in the constructs and methods of the present invention.

In an embodiment, the above-mentioned immobilizing moiety is a GST tag and said solid support is a Gutathione-containing solid support, in a further embodiment Glutathione Sepharose™.

In embodiments, binding to the solid support (on which the above-mentioned construct is bound) may for example be achieved by batch treatment or column chromatography. Batch treatment typically entails combining the sample (containing the RNA of interest) with the solid support in a vessel to allow binding of the RNA, mixing, separating the solid support (e.g., by centrifugation), removing the liquid phase, washing, separating the solid support (e.g., re-centrifuging), adding an elution buffer, separating the solid support (e.g., re-centrifuging) and removing the eluate. Column chromatography typically entails packing the solid support onto a chromatography column, passing the sample (containing the RNA of interest) through the column to allow binding of the RNA, passing a wash buffer through the column and subsequently an elution buffer to collect the bound material. Hybrid approaches may also be used, for example binding via a batch method followed by packing the solid support with the bound target RNA onto a column, followed by washing and elution on the column. In embodiments, the batch method can also be combined with the use of spin cups and Steriflip™ filter units, which typically improve resin recovery.

In embodiments, the above-mentioned construct further comprises one or more additional domains. In other embodiments, the N- and/or C-terminal end(s) of the construct is/are modified.

The above-mentioned peptide/polypeptide construct may be produced by expression in a host cell comprising a nucleic acid encoding the construct (recombinant expression) or by chemical synthesis (e.g., solid-phase peptide synthesis). Peptides/polypeptides can be readily synthesized by automated solid phase procedures well known in the art. Suitable syntheses can be performed by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the peptides may be prepared by way of segment condensation, as described, for example, in Liu et al., Tetrahedron Lett. 37: 933-936, 1996; Baca et al., J. Am. Chem. Soc. 117: 1881-1887, 1995; Tam et al., Int. J. Peptide Protein Res. 45: 209-216, 1995; Schnolzer and Kent, Science 256: 221-225, 1992; Liu and Tam, J. Am. Chem. Soc. 116: 4149-4153, 1994; Liu and Tam, Proc. Natl. Acad. Sci. USA 91: 6584-6588, 1994; and Yamashiro and Li, Int. J. Peptide Protein Res. 31: 322-334, 1988). Other methods useful for synthesizing the peptides are described in Nakagawa et al., J. Am. Chem. Soc. 107: 7087-7092, 1985. Commercial providers of polypeptide/peptide synthetic services may also be used to prepare synthetic polypeptides/peptides in the D- or L-configuration. Such providers include, for example, Advanced ChemTech (Louisville, Ky.), Applied Biosystems (Foster City, Calif.), Anaspec (San Jose, Calif.), and Cell Essentials (Boston, Mass.).

Polypeptides and peptides comprising naturally occurring amino acids encoded by the genetic code may also be prepared using recombinant DNA technology using standard methods. Polypeptides and peptides produced by recombinant technology may be modified (e.g., N-terminal acylation [e.g., acetylation], C-terminal amidation) using methods well known in the art. Accordingly, in another aspect, the invention further provides a nucleic acid encoding the above-mentioned construct. The invention also provides a vector comprising the above-mentioned nucleic acid. In yet another aspect, the present invention provides a cell (e.g., a host cell) comprising the above-mentioned nucleic acid and/or vector. The invention further provides a recombinant expression system, vectors and host cells, such as those described above, for the expression/production of the above-mentioned construct, using for example culture media, production, isolation and purification methods well known in the art.

Such vectors comprise a nucleic acid sequence capable of encoding the construct operably linked to one or more transcriptional regulatory sequence(s). Nucleic acids may be introduced into cells for expression using standard recombinant techniques for stable or transient expression. Nucleic acid molecules of the invention may include any chain of two or more nucleotides including naturally occurring or non-naturally occurring nucleotides or nucleotide analogues.

“Recombinant expression” refers to the production of a peptide or polypeptide by recombinant techniques, wherein generally, a nucleic acid encoding a peptide or polypeptide is inserted into a suitable expression vector which is in turn used to transform/transfect a host cell to produce the protein. The term “recombinant” when made in reference to a protein or a polypeptide refers to a peptide, polypeptide or protein molecule which is expressed using a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e., by human intervention. Recombinant nucleic acid constructs may for example be introduced into a host cell by transformation/transfection. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

The term “vector” or “plasmid” refers to a nucleic acid molecule, which is capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”.

A recombinant expression vector/plasmid of the present invention can be constructed by standard techniques known to one of ordinary skill in the art and found, for example, in Sambrook et al. (1989) in Molecular Cloning: A Laboratory Manual. A variety of strategies are available for ligating fragments of DNA, the choice of which depends on the nature of the termini of the DNA fragments and can be readily determined by persons skilled in the art. The vectors may also contain other sequence elements to facilitate vector propagation and selection in bacteria and host cells. In addition, the vectors of the present invention may comprise a sequence of nucleotides for one or more restriction endonuclease sites. Coding sequences such as for selectable markers and reporter genes are well known to persons skilled in the art.

A recombinant expression vector comprising a nucleic acid of the present invention may be introduced into a host cell, which may include a living cell capable of expressing the protein coding region from the defined recombinant expression vector. The living cell may include both a cultured cell and a cell within a living organism. Accordingly, the invention also provides a host cell (e.g., an isolated host cell) containing the recombinant expression vectors of the invention. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector/plasmid DNA can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (Molecular Cloning: A Laboratory Manual, 2^{nd} Edition, Cold Spring Harbor Laboratory press (1989)), and other laboratory manuals. Methods for introducing DNA into mammalian cells in vivo are also known, and may be used to deliver the vector DNA of the invention to a subject for gene therapy.

“Transcriptional regulatory sequence/element” is a generic term that refers to DNA sequences, such as initiation and termination signals, enhancers, and promoters, splicing signals, polyadenylation signals which induce or control transcription of protein coding sequences with which they are operably linked. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous.

As used herein, the term “transfection” or “transformation” generally refers to the introduction of a nucleic acid, e.g., via an expression vector/plasmid, into a recipient cell by nucleic acid-mediated gene transfer.

A cell (e.g., a host cell or indicator cell), tissue, organ, or organism into which has been introduced a foreign nucleic acid (e.g., exogenous or heterologous DNA [e.g. a DNA construct]), is considered “transformed”, “transfected”, or “transgenic”. A transgenic or transformed cell or organism also includes progeny of the cell or organism and progeny produced from a breeding program employing a transgenic organism as a parent and exhibiting an altered phenotype resulting from the presence of a recombinant nucleic acid construct. A transgenic organism is therefore an organism that has been transformed with a heterologous nucleic acid, or the progeny of such an organism that includes the transgene. The introduced DNA may be integrated into chromosomal DNA of the cell's genome, or alternatively may be maintained episomally (e.g., on a plasmid). Methods of transfection are well known in the art (see for example, Sambrook et al., 1989, supra; Ausubel et al., 1994 supra).

For stable transfection of mammalian cells, it is known that, depending upon the expression vector/plasmid and transfection technique used, only a small fraction of cells may integrate the foreign DNA into their genome. In order to identify and select these integrants, a gene that encodes a selectable marker (such as resistance to antibiotics) may be introduced into the host cells along with the gene of interest. As used herein, the term “selectable marker” is used broadly to refer to markers which confer an identifiable trait to the indicator cell. Non-limiting example of selectable markers include markers affecting viability, metabolism, proliferation, morphology and the like. Preferred selectable markers include those that confer resistance to drugs, such as G418, hygromycin and methotrexate. Nucleic acids encoding a selectable marker may be introduced into a host cell on the same vector as that encoding the peptide compound or may be introduced on a separate vector. Cells stably transfected with the introduced nucleic acid may be identified by drug selection (cells that have incorporated the selectable marker gene will survive, while the other cells die).

The polypeptide of the invention can be purified by many techniques well known in the art, such as reverse phase chromatography, high performance liquid chromatography (HPLC), ion exchange chromatography, size exclusion chromatography, affinity chromatography, gel electrophoresis, and the like. The actual conditions used to purify a particular peptide or peptide analog will depend, in part, on synthesis strategy and on factors such as net charge, hydrophobicity, hydrophilicity, and the like, and will be apparent to those of ordinary skill in the art. For affinity chromatography purification, any antibody which specifically binds the peptide or polypeptide may for example be used.

In an embodiment, the above-mentioned construct is substantially pure. A compound is “substantially pure” when it is separated from the components that naturally accompany it. Typically, a compound is substantially pure when it is at least 60%, more generally 75%, preferably over 90% and more preferably over 95%, by weight, of the total material in a sample. Thus, for example, a polypeptide that is chemically synthesized or produced by recombinant technology will generally be substantially free from its naturally associated components. A nucleic acid molecule is substantially pure when it is not immediately contiguous with (i.e., covalently linked to) the coding sequences with which it is normally contiguous in the naturally occurring genome of the organism from which the DNA of the invention is derived. A substantially pure compound can be obtained, for example, by extraction from a natural source; by expression of a recombinant nucleic acid molecule encoding a polypeptide compound; or by chemical synthesis. Purity can be measured using any appropriate method such as column chromatography, gel electrophoresis, HPLC, etc.

The above-mentioned construct may be useful for a variety of applications, and more particularly applications in which immobilization of RNA is useful/desirable. Such applications include, for example, RNA enrichment/purification (i.e., to enrich/purify a RNA of interest or target RNA), as well as site-specific (or segmental) labelling of RNA (with fluorophores or other probes such as isotopes).

Accordingly, in further aspects, the present invention further provides:
a method for immobilizing a target RNA, said method comprising:
   (a) providing a bacteriophage boxB-comprising target RNA comprising a bacteriophage boxB RNA and the target RNA; and
   (b) contacting the bacteriophage boxB-comprising target RNA of (a) with the above-mentioned construct bound to a solid support;
a method for immobilizing a target RNA, said method comprising:
   (a) providing a bacteriophage boxB-comprising target RNA comprising a bacteriophage boxB RNA and the target RNA; and
   (b) contacting the bacteriophage boxB-comprising target RNA of (a) with the above-mentioned construct, thereby to obtain a complex comprising the bacteriophage boxB-comprising target RNA bound to the construct;
   (c) contacting the complex with a solid support comprising a ligand capable of binding to the immobilizing moiety;
a method for purifying a target RNA, said method comprising:
   (a) providing an affinity tag-comprising target RNA comprising an affinity tag and the target RNA, wherein said affinity tag comprises a bacteriophage boxB sequence and an activatable ribozyme sequence;
   (b) contacting the affinity tag-comprising target RNA of (a) with the above-mentioned construct bound to a solid support;
   (c) inducing activation of said activatable ribozyme; and
   (d) collecting said target RNA;
a method for purifying a target RNA, said method comprising:
   (a) providing an affinity tag-comprising target RNA comprising an affinity tag and the target RNA, wherein said affinity tag comprises a bacteriophage boxB sequence and an activatable ribozyme sequence;
   (b) contacting the affinity tag-comprising target RNA of (a) with the above-mentioned construct thereby to obtain a complex comprising the affinity tag-comprising target RNA bound to the construct;
   (c) contacting the complex with a solid support comprising a ligand capable of binding to the immobilizing moiety;
   (d) inducing activation of said activatable ribozyme; and
   (e) collecting said target RNA.

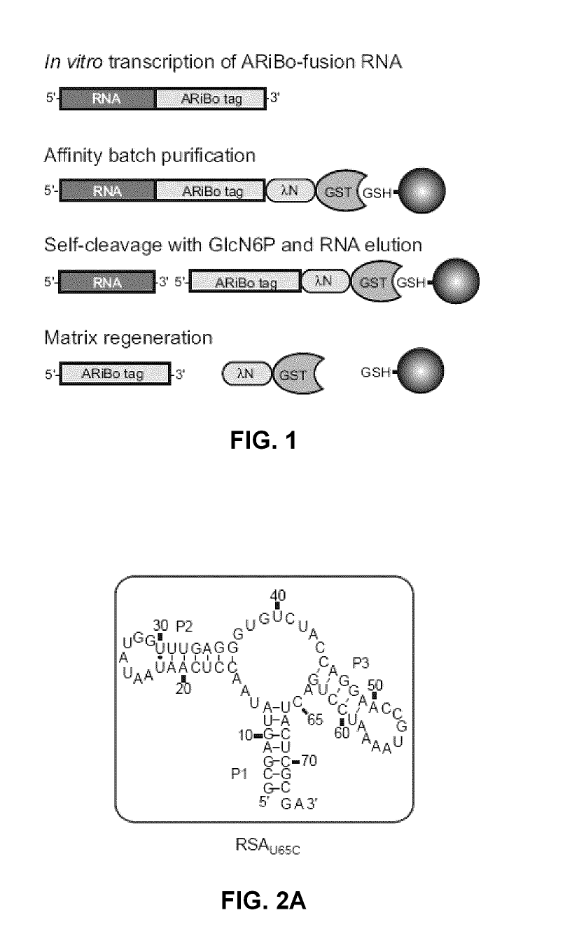

Activatable ribozyme as used herein refers to a ribonucleic acid molecule capable of catalyzing the breaking of a phosphodiester bond at a specific site within the affinity tag of the above-mentioned affinity tag-comprising target RNA following activation (the activatable ribozyme exhibit no or very low catalytic activity in the absence of activation). The breaking of the phosphodiester bond allows the target RNA to be released from the affinity tag (which remains bound to the solid support, as exemplified in FIG. 1), thereby facilitating elution and purification of the target RNA. An activatable ribozyme may be activated by a variety of ways, including by effector molecules and/or other means such as radiation (e.g., light radiation, UV radiation, IR radiation), as well as temperature and/or pH changes.

Activatable ribozymes are well known in the art and include, for example, those described in Koizumi et al., Nature Struct. Biol. 6(11), 1062-1071 (1999) which are activable by specific nucleoside 3′,5′-cyclic monophosphate compounds such as cGMP or cAMP, those described in Grate and Wilson, Proc. Nat. Acad. Sci 96: 6131-6136 (1999) (a malachite green (MG)-tagged RNA that cleaves upon laser irradiation) as well as the Glucosamine-6-phosphate activated (GlmS) ribozyme which is activated by glucosamine-6-phosphate (GlcN6P). Other examples of suitable activatable ribozymes include virus-derived activatable ribozymes such as activatable hepatitis delta virus ribozyme (HEN) (Shih et al., Annual Review of Biochemistry 71: 887-917 (2002)). In an embodiment, the above-mentioned activatable ribozyme sequence is a sequence of a glmS ribozyme, which are typically produced by Gram-positive bacteria such as Bacillus subtilis and Bacillus anthracis (Roth, A. et al. (2006) RNA, 12, 607-619). In a further embodiment, the above-mentioned glmS ribozyme is a Bacillus anthracis glmS ribozyme sequence. In a further embodiment, the above-mentioned glmS ribozyme sequence is encoded by one of the following DNA sequences:

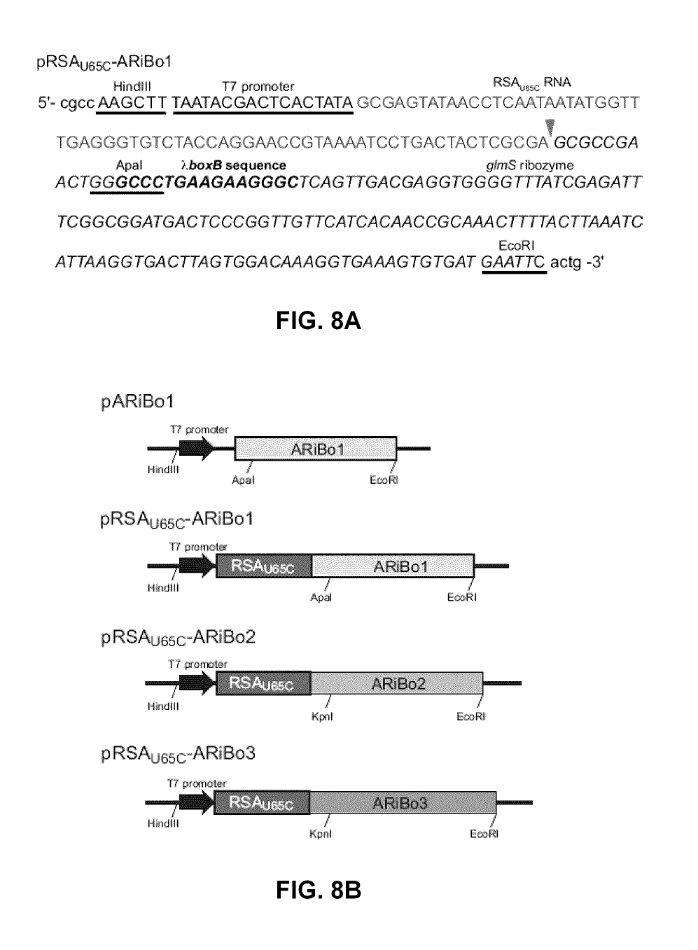

(FIG. 8A, SEQ ID NO: 64)
3′-CGCGGCTTGACCCGGGACTTCTTCCCGAGTCAACTGCTCCACCCCAA
ATAGCTCTAAAGCCGCCTACTGAGGGCCAACAAGTAGTGTTGGCGTTTGA
AAATGAATTTAGTAATTCCACTGAATCACCTGTTTCCACTTTCACACTAC
T5′;
or
(Batey et Kieft, supra, SEQ ID NO: 65)
3′-CGCGGGCTTGATGGCCATGGCCATCAACTGCTCCTACCTCCAATAGC
TTAAAAGCCGCCTACGGAGGGCCGACTCACACGTCTAGTGTCGGCATTCC
TAAAGAAGTTTGGTTCCCCCACTGAGGAACTTGTTTCTCTTTAGTGTACT
AGA5′.

Moreover, methods for developing ribozymes capable of being activated by specific effector molecules are well known (see, for example, U.S. Pat. No. 6,630,306; Winkler et al., Nature 428: 281-286 (2004); Koisumi et al., 1999, supra; and Seetherman et al., Nature Biotech. 19: 336-341 (2001)). Notably, activatable ribozymes may be generated by combining a ribozyme and a riboswitch, as described in Chen and Elington, PLoS Comput Biol 2009 5(12): e1000620.

The above-mentioned bacteriophage boxB sequence and/or activatable ribozyme sequence may be incorporated at the 5′ or 3′ end of the target, or within the target RNA. In an embodiment, the above-mentioned bacteriophage boxB sequence and/or activatable ribozyme sequence is/are incorporated at the 3′ end of the target RNA.

In another embodiment, the above-mentioned method further comprises incorporating a linker at the 3′ end of said target RNA (e.g., between the 3′ end of the target RNA and the affinity tag sequence). In an embodiment, the linker is a short linker (e.g., 10 nucleotides or less) comprising any nucleotides or combinations thereof. In an embodiment, the linker is a linker of 5 nucleotides or less, and in a further embodiment of 1 or 2 nucleotides. In a further embodiment, the linker is GA, GG, GC, GU or A.

The above-mentioned bacteriophage boxB sequence may be incorporated before (i.e. at the 5′ end), after (i.e. at the 3′ end) or within the activatable ribozyme sequence. In an embodiment, the above-mentioned bacteriophage boxB sequence is incorporated after (i.e. at the 3′ end) the activatable ribozyme sequence. In another embodiment, the above-mentioned bacteriophage boxB sequence is incorporated into a stem-loop of the activatable ribozyme sequence, in a further embodiment into the variable apical P1 stem-loop of the activatable ribozyme sequence.

In an embodiment, the above-mentioned target RNA, bacteriophage boxB-comprising target RNA and/or affinity tag-comprising target RNA is/are produced by recombinant technology, for example using an expression vector/plasmid comprising sequence encoding the above-mentioned target RNA, bacteriophage boxB-comprising target RNA and/or affinity tag-comprising target RNA. The expression vector may include appropriate restriction site sequences, transcription control sequence, sequences encoding antibiotic resistance genes, etc. The expression vector may be transcribed in vitro or transformed into an appropriate bacterial strain (E. coli) or any appropriate cell types (e.g., yeast cells, mammalian cells), such that the target RNA, bacteriophage boxB-comprising target RNA and/or affinity tag-comprising target RNA can be produced. Following the growth and expression procedure, the host cells may be lysed and the lysate passed over a solid support (e.g., an affinity resin that binds to the immobilizing moiety) to capture the expressed target RNA.

In an embodiment, when contacting the bacteriophage boxB-comprising target RNA with the polypeptide construct, the [polypeptide construct]:[bacteriophage boxB-comprising target RNA] ratio is from about 10:1 to about 2:1, in a further embodiment from about 8:1 to about 3:1, in yet a further embodiment from about 6:1 to about 4:1, e.g., 5:1.

In an embodiment, the above-mentioned method results in a RNA yield of about 40% or more, in a further embodiment about 45% or more, in a further embodiment about 50% or more, in a further embodiment about 55% or more, in a further embodiment about 60% or more.

In an embodiment, the above-mentioned method results in a RNA purity of about 90% or more, in a further embodiment about 95% or more, in a further embodiment about 96% or more, in a further embodiment about 97% or more, in a further embodiment about 98% or more, in a further embodiment about 99% or more, in a further embodiment about 99.5% or more.

In embodiments, the above-mentioned method further comprises regenerating the solid support/matrix after elution of the target RNA, by removing or eluting the affinity tag and the construct (which comprises the immobilizing moiety, the linker and the boxB RNA binding peptide) from the solid support, as illustrated in FIG. 1. This may be achieved, for example, by contacting the solid support with a saline solution (e.g., a NaCl solution, such as a concentrated (2.5M) NaCl solution) to elute the affinity tag. The construct may next be removed/eluted from the solid support using methods well known in the art. For example, if the immobilizing moiety is a Glutathione S-transferase (GST) polypeptide and the solid support is a Glutathione Sepharose™ bead, the immobilizing moiety may be dissociated from the solid support by contacting the solid support with a glutathione (GSH) solution (e.g., a 20 mM GSH solution), thereby eluting the construct. If the immobilizing moiety is an His-tag and the solid support is a metal-containing matrix/bead (e.g., a nickel matrix), the immobilizing moiety may be dissociated from the solid support by contacting the solid support with an imidazole solution (e.g., a 10 mM to 1M imidazole solution), thereby eluting the construct.

The above-mentioned method may further comprise one or more washing steps.

The target RNA (or RNA of interest) may be any type of RNA (naturally occurring or not) including messenger RNA (mRNA), UTRs, transfer RNA (tRNA), ribosomal RNA (rRNA), transfer-messenger RNA (tmRNA), microRNA (miRNA), small interfering RNA (sRNA), riboswitch RNA, small nuclear RNA (snRNA), small nucleolar RNA (snoRNA), trans-acting sRNA (tasiRNA), repeat-associated sRNA (rasiRNA), small temporary RNA (stRNA), tiny non-coding RNA (tncRNA), small scan RNA (snRNA), and small modulatory RNA (smRNA).

In an embodiment, one or more steps of the above-mentioned method are performed in the absence of Tris (tris(hydroxymethyl)aminomethane) buffer. In another embodiment, one or more steps of the above-mentioned method are performed under RNAse-free conditions.

In another aspect, the present invention provides a kit for immobilizing/purifying a bacteriophage boxB-comprising RNA, said kit comprising the above-mentioned construct. In an embodiment, the above-mentioned kit further comprises the above-mentioned solid support. In an embodiment, the above-mentioned kit further comprises a nucleic acid construct comprising a sequence encoding a boxB RNA and/or a nucleic acid construct comprising a sequence encoding the above-noted activatable ribozyme (in an embodiment both such sequences may be comprised in the same nucleic acid construct or vector). Such kits may further comprise, for example, control samples, containers, reaction or purification vessels, as well as one or more reagents useful for RNA immobilization and/or elution (buffers, solution, enzymes, columns, plates, tubes), for example an agent capable of activating the activatable ribozyme. The kit may further comprise materials and reagents useful for incorporating a bacteriophage boxB sequence and/or an affinity tag into a target RNA sequence. For example, the kit may comprises one or more DNA plasmids/vectors comprising sequences encoding the bacteriophage boxB and/or the affinity tag and in which a DNA sequence encoding a target RNA may be incorporated at one or more specific sites (e.g., using restriction enzymes) to generate a bacteriophage boxB- and/or affinity tag-comprising RNA.

### MODE(S) FOR CARRYING OUT THE INVENTION

The present invention is illustrated in further details by the following non-limiting examples.

### Example 1

### Materials and Methods

Cloning of Protein Expression Vectors.

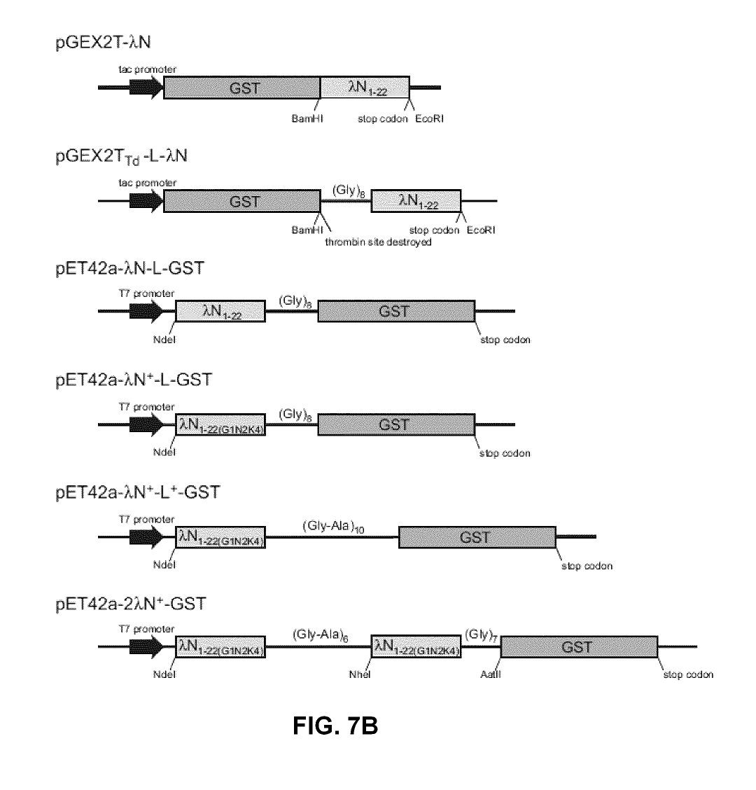

All vectors used for protein expression are illustrated in FIG. 7B. The pGEX2T-λN plasmid was described previously (Mogridge, J. et al. (1998) Mol Cell, 1, 265-275). For pGEX2T_{Td}-L-λN, a DNA fragment coding for G₈-λN₁₋₂₂ was inserted at the BamH1 and EcoR1 sites of pGEX2T (GE Healthcare), and the thrombin cleavage-site was destroyed (LVPRGS to LVPGGS) by mutagenesis. All mutageneses reported here were conducted according to the Stratagene QuikChangell™ site-directed mutagenesis kit protocols. For pET42a-λN-L-GST, a DNA fragment coding for λN₁₋₂₂-G₈ was inserted into the NdeI site of the pET42a vector (Novagen), and a stop codon was created by mutagenesis at the end of the GST-coding sequence. Mutagenesis of pET42a-λN-L-GST was carried out to introduce the M1G/D2N/Q4K mutations (Austin et al., 2002, supra) within the coding sequence of the λN peptide, which yielded pET42a-λN⁺-L-GST. Mutagenesis of this later vector was carried out to change the G₈-linker sequence to (GA)₁₀ and thereby obtain pET42a-λN⁺-L⁺-GST. The pET42a-2λN⁺-GST plasmid was generated as a by-product of a multi-step cloning procedure. First, NheI and AatII restriction sites were introduced by mutagenesis just upstream of the GST-coding sequence of pET42a-λN⁺-L⁺-GST. A PCR fragment generated from pET42a-λN⁺-L-GST and coding for λN⁺-G₈ was inserted within the new NheI and AatII sites. All sequences were verified by DNA sequencing.

Expression and Purification of GST-Fusion Proteins.

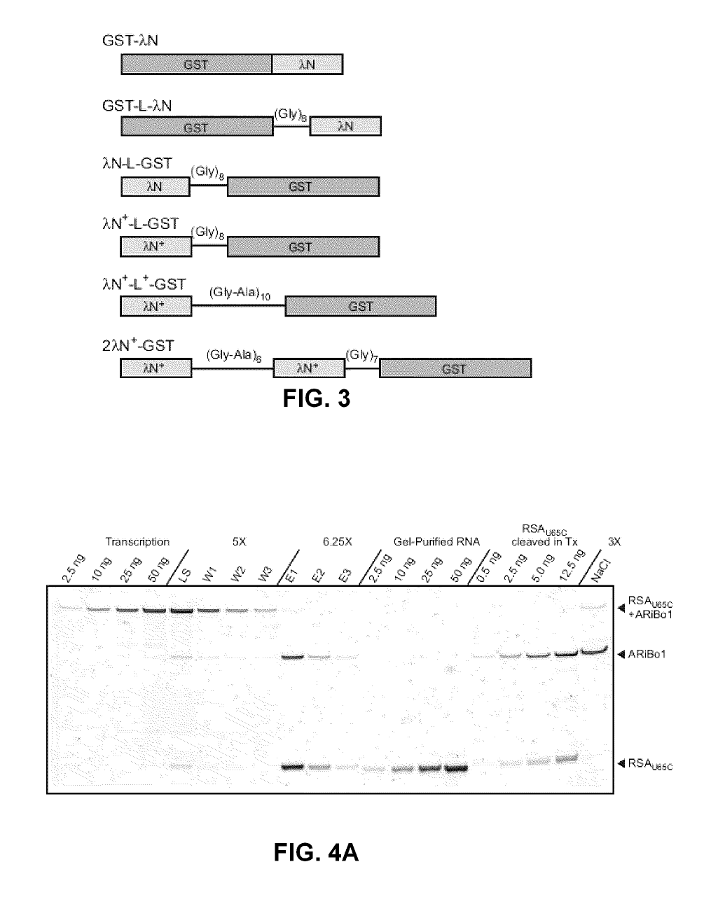

The pGEX2T-based plasmids used for expression of GST-λN and GST-L-λN were transformed into BL21 cells, whereas the pET42a-based plasmids used for expression of λN-L-GST, λN⁺-L⁺-GST and 2λN⁺-GST were transformed into BL21(DE3) cells (FIG. 3). All cells were grown at 37° C. in Luria-Bertani media (4 L), and protein expression was induced with 1 mM isopropyl-β-D-1-thiogalactopyranoside (IPTG) for 4 hr at 30° C. The cells were harvested by centrifugation and resuspended in Homogenisation buffer [20 mM Tris pH=7.4, 1 M NaCl, 1 mM DTT, 0.2 mM EDTA and 0.15% w/v protease inhibitor cocktail (Sigma-Aldrich)]. The cells were lysed by French press, sonicated 10 sec and centrifuged at 138 000 g for 1 hr (4° C.). The supernatant was incubated for 1 hr at 4° C. with GSH-Sepharose™ 4B resin (GE Healthcare). The resin was washed 2 times with Wash buffer (Homogenisation buffer with 2 M urea), and then 2 times with PBS buffer (10 mM Na₂HPO₄, 2 mM KH₂PO₄, 2.7 mM KCl, 140 mM NaCl, and pH=7.4). The GST-fusion proteins were eluted from the bound resin by two incubations of 15 min at room temperature with 20 mM reduced L-glutathione pH=8.0 (Sigma-Aldrich). The supernatant was dialyzed overnight at 4° C. in FPLC-A buffer (20 mM sodium phosphate pH=7.4, 1 mM EDTA and 1 mM DTT) and then applied to an SP Sepharose™ high performance column (GE Healthcare; 100 mL bed volume) equilibrated with FPLC-A buffer. The proteins were eluted from the column using a gradient (from 0 to 100% over 525 mL) of FPLC-B buffer (FPLC-A with 2 M NaCl). The pooled fractions containing the protein of interest were dialyzed into water and then lyophilized. The high purity (≧95%) and correct mass (with error ≦±1.4 Da) of all purified proteins were verified by SDS-PAGE and mass spectrometry, respectively.

Cloning of the pARiBo-Based Plasmids.

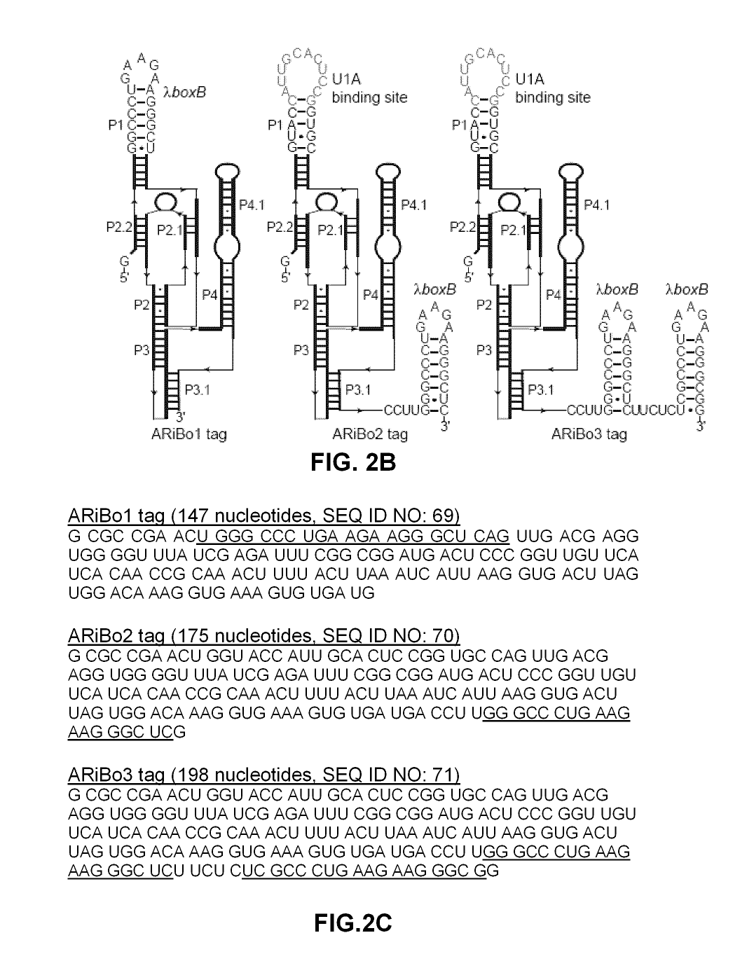

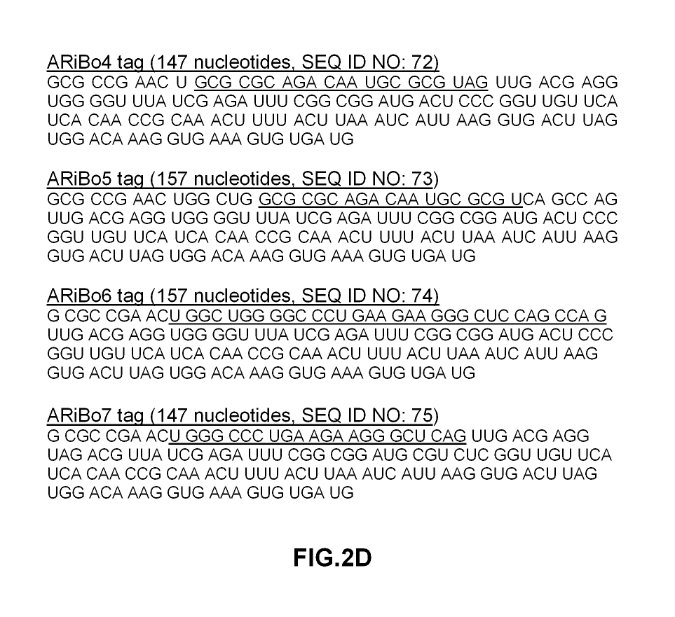

All transcription vectors are depicted in FIGS. 8A and 8B. To generate the pARiBo1 plasmid, a DNA fragment was generated which contains the T7 promoter (5′-TAATACGACTCACTATA-3′) and codes for a 5′-GGCGAA-3′ sequence followed by the ARiBo1 RNA (FIG. 2B). This fragment was inserted between the HindIII and EcoR1 sites of the pTZ19R-derived pTR-4 vector (Rastogi, T. and Collins, R. A. (1998) J Mol Biol, 277, 215-224). The sequence of the ARiBo1 RNA was designed to incorporate an ApaI restriction site in the P1 helix (FIG. 2B). For the pRSA_{U65C}-ARiBo1 plasmid, a DNA fragment containing the T7 promoter and coding for the RSA_{U65C} RNA was first generated by PCR amplification of the pRSA_{U65C}-VS plasmid (Delfosse, V. et al. (2010) Nucleic Acids Res, 38, 2057-2068) and then inserted between the HindIII and ApaI sites of the pARiBo1 plasmid. The pRSA_{U65C}-ARiBo2 and pRSA_{U65C}-ARiBo3 plasmids (FIG. 2B) were obtained by mutagenesis of the pRSA_{U65C}-ARiBo1 plasmid using the QuikChangell™ site-directed mutagenesis procedure. The sequences of the ARiBo2 and ARiBo3 RNAs were designed to incorporate a KpnI restriction site in the P1 helix (FIG. 2B). All sequences were verified by DNA sequencing.

In Vitro Transcription of ARiBo-Fused RNAs.

Large-scale preparations (˜2-3 mg) of plasmid DNA template were typically obtained by growing 0.5 L of plasmid-transformed DH5α cells (Invitrogen), purifying the plasmid using the QIAGEN™ Plasmid Mega Kit and linearizing it overnight with EcoRI (New England Biolabs). The ARiBo-fused RNAs were transcribed at 37° C. for 3 h using the following reaction conditions: 40 mM HEPES pH=8.0, 50 mM DTT, 0.1% Triton™-X-100, 1 mM spermidine, 4 mM ATP, 4 mM CTP, 4 mM UTP, 8 mM GTP, 25 mM MgCl₂, 60 μg/ml T7 RNA polymerase, 3 U/ml RNAsin™ Ribonulease Inhibitor (Promega) and 80 μg/ml of linearized plasmid DNA template. Transcription reactions were stopped by adding 50 mM EDTA and stored at −20° C.

Small-Scale Affinity Batch Purification of ARiBo-Fused RNAs.

For typical small-scale purifications, 35 nmol of GST/λN-fusion protein was first added to a small transcription volume (˜140 μl for RSA_{U65C}) that corresponds to 7 nmol of ARiBo-fused RNA, and the total volume was adjusted to 800 μl with Equilibration buffer (50 mM HEPES pH=7.5). The RNA-protein mix was then incubated for 15 min in a 1.5-ml conical tube. Unless otherwise mentioned, all incubations were done with gentle rotation at room temperature. In a separate tube, 325 μl of GSH-Sepharose™ 4B resin slurry (GE Healthcare) was washed twice with 800 μl of PBS buffer. The RNA-protein mix was added to the washed resin and incubated for 15 min, centrifuged 1 min at 1500 g, and the load supernatant was kept for quantitative analysis on gel (LS). The pelleted resin was washed three times with 800 μl Equilibration buffer. These and all subsequent washes involved incubation for 5 min and centrifugation for 1 min at 1500 g. The wash supernatants were kept for quantitative analysis (W1, W2, W3). Elution of the desired RNA (RSA_{U65C}) was induced by leaving the pelleted resin at 37° C. for 10 min in 800 μl Elution buffer (20 mM Tris buffer pH=7.6, 10 mM MgCl₂ and 500 μM GlcN6P), and transferring for 5 min at room temperature prior to centrifugation. The elution supernatant contained the RNA of interest (E1). The pelleted resin was washed two times with 800 μl Equilibration buffer, and the elution-wash supernatants were kept for quantitative analysis (E2, E3). To prevent loss of resins during the wash and elution steps, all supernatants were centrifuged for 1 min at 1500 g and the minute amount of pelleted resin was mixed with the buffer used for the next purification step. To remove RNA left on the resin after elution, the pelleted resin was washed with 800 μl of 2.5 M NaCl. The supernatant was kept for quantitative analysis (NaCl). For complete matrix regeneration, the GSH-Sepharose™ resin was subsequently washed with PBS, 20 mM L-glutathione pH=8 in PBS and then with 20% ethanol for storage.

Quantitative Analysis of Small-Scale Affinity Batch Purifications.

For quantitative analysis, each small-scale affinity batch purification was performed at least three times, and purifications made from the same ARiBo-fused RSA_{U65C} precursor were performed from the same transcription reaction. Aliquots from the various steps of purification were analyzed by denaturing-gel electrophoresis. Care was taken to load gels with sample volumes corresponding to precise amounts of RSA_{U65C}-ARiBo-fusion RNA present in the transcription reaction. The gels were stained for 10 min in a SYBR™ Gold (Invitrogen) solution [1:10000 dilution in TBE buffer (200 mM Tris-Base, 200 mM boric acid and 4 mM EDTA)] and scanned on a Molecular FX™ densitometer (Bio-Rad). The band intensities were analyzed using the QuantityOne™ software (version 4.4.1 from Bio-Rad).

For each gel, several control lanes were loaded with known amounts of RNA to derive three standard curves that were used to determine the quantity in ng of RSA_{U65C} (N_{RNA}), ARiBo tag (N_{ARiBo}) and RSA_{U65C}-ARiBo fusion RNA (N_{Fusion}) at each purification step. For the RSA_{U65C} standard curve, the quantities of RNA loaded on the gel were obtained from OD₂₆₀ measurements. For the standard curves of the ARiBo-tag and the RSA_{U65C}-ARiBo-fusion RNAs, the quantities of RNA loaded on the gel were calculated from the quantity of RSA_{U65C} detected in transcription reactions treated with GlcN6P to achieve ≧99% ARiBo tag cleavage.

The percentage of unbound RNA was calculated using the equation [(ΣN_{Fusion})/I_{Fusion}]*100%, where ΣN_{Fusion} represents the total amount of fusion RNA (ng) detected in lanes LS, W1, W2 and W3, and I_{Fusion} represents the input of the same RNA in equivalent volumes of affinity batch purification (250 ng). The percentage of unbound RNA is given as a minimum value, since it is only based on the amount of fusion RNA that migrates as such on the gel; slower migrating species have been observed in certain cases and probably represent some forms of RNA aggregates, but were not quantified.

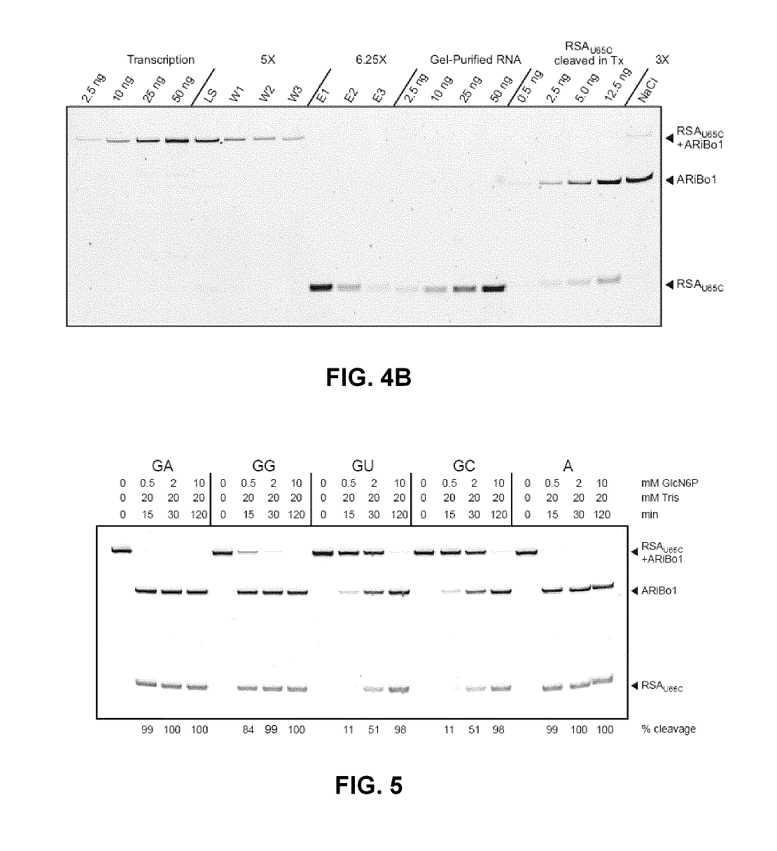

The percentage of cleavage in solution was determined from a control lane in which the transcription reaction was treated with GlcN6P (FIGS. 4A and 4B, lane 19) using the equation: {(N_{ARiBo}/nt_{ARiBo})/[(N_{ARiBo}/nt_{ARiBo})/(N_{Fusion}/nt_{Fusion})]}*100%, where nt_{ARiBo} and nt_{Fusion} represent the number of nucleotides for the ARiBo-tag and fusion RNAs, respectively. The same equation was used to calculate the percentage of cleavage on the resin, although this was derived from the NaCl lane (FIGS. 4A and 4B, lane 20).

The percentage of RNA eluted was calculated using the equation: [(ΣN_{RNA})/I_{RNA}]*100%, where ΣN_{RNA} represents the total amount of RSA_{U65C} (ng) detected in lanes E1, E2 and E3, and I_{RNA} represents the calculated amount of RSA_{U65C} expected from 100% cleavage in equivalent volumes of transcription (100 ng).

The percentage of RNA purity was calculated from the E1 lane (FIGS. 4A and 4B, lane 9) using the equation: [N_{RNA}/(N_{RNA}+N_{ARiBo})]*100%.

Large-Scale Affinity Batch Purification of ARiBo-Fused RNAs.

Large-scale purifications were processed in 50-mL conical tubes, similarly to the small-scale purifications, but increasing all volumes 30 times (25-ml wash and elution buffers). In addition, an alkaline phosphatase step was inserted between the first (W1) and second washes (W2). This consisted of a 4-hr incubation at 37° C. in 25 ml of CIP buffer (50 mM HEPES pH=8.5 and 0.1 mM EDTA) with 130 U of calf intestine alkaline phosphatase (Roche) per pmole of RNA, followed by a 5-min incubation at room temperature prior to centrifugation. The supernatant was kept for analysis (CIP). The purified RNA (E1, E2 and E3 fractions) was concentrated with Amicon™ Ultra-15 centrifugal filter devices (Millipore) and exchanged in NMR buffer (10 mM sodium cacodylate pH=6.5, 50 mM KCl, 5 mM MgCl₂, 0.05 mM NaN₃ in 90% H₂O/10% D₂O).

In Vitro Transcription of RNA and Purification by Denaturing Gel Electrophoresis.

RSA_{U65C} was also synthesized as an RSA_{U65C}-VS precursor containing a Varkud Satellite (VS) ribozyme substrate at its 3′-end. In vitro transcription and purification of RSA_{U65C} by denaturing-gel electrophoresis was performed as described previously (Delfosse et al., 2010, supra), except that the HPLC purification step using a DNA-Pac100™ column heated at 65° C. was replaced by gravity-flow anion-exchange chromatography with DEAE-Sephacel™ at room temperature.

NMR Spectroscopy Studies.

The 1D ¹H flip-back watergate spectra were collected at 15° C. on a Varian^{Unity} INOVA™ 500 MHz spectrometer equipped with a pulse-field gradient unit and an actively-shielded z gradient ¹H{¹³C/¹⁵N} triple resonance probe.

### Example 2

### General Scheme for Affinity Purification of RNA Using ARiBo Tags

To develop an efficient affinity-purification procedure that maximizes RNA yield and purity, the GST/λN-fusion proteins attached to a GSH-Sepharose™ matrix was used. The natural λN and its cognate boxB RNA form a very stable and specific interaction (K_{D}˜2-20 nM), and increased stability can be obtained using engineered λN peptides (K_{D}≧10 pM) (Austin et al., 2002, supra; Legault, P. et al. (1998) Cell, 93, 289-299). The GST/GSH-Sepharose™ system is one of the most affordable and commonly used affinity methods for purification of recombinant proteins expressed in Escherichia coli (E. coli). The GST/GSH-Sepharose™ interaction is compatible with all commonly used aqueous buffers, yet easily reversible by addition of free glutathione. For RNA elution, the glmS ribozyme from B. anthracis (Winkler, W. C., et al. (2004) Nature, 428, 281-286; Wilkinson, S. R. and Been, M. D. (2005) RNA, 11, 1788-1794; Cochrane, J. C. et al. (2007) Chem Biol, 14, 97-105) was used. The glmS ribozyme self-cleaves quickly and efficiently when activated by Glc6NP, and displays very low background activity in the absence of Glc6NP, Tris and related compounds (Winkler, W. C., et al. (2004), supra; Cochrane, J. C. et al. (2007), supra; McCarthy, T. J. et al. (2005) Chem Biol, 12, 1221-1226; Roth, A. et al. (2006) supra). This activatable ribozyme was combined with the λBoxB RNA to create a novel affinity tag, termed the ARiBo tag.

The general strategy of the procedure utilized in the studies described herein is outlined in FIG. 1. The RNA of interest is first transcribed with an ARiBo tag at its 3′-end. The ARiBo-fusion RNA is then bound to a GST/λN-fusion protein, and the resulting complex is captured on GSH-Sepharose™ resin. After washing to remove impurities, the RNA is eluted by self-cleavage of the glmS ribozyme following the addition of GlcN6P to activate the ribozyme. As needed, the resin can be regenerated using 2.5 M NaCl to remove the affinity tag and 20 mM GSH to liberate the GST/λN-fusion protein.

### Example 3

### Development of an Optimal Affinity Batch Purification Method

Although affinity purifications are often performed in a gravity-column or spin-column format, a batch method was used in the studies described herein. The batch format is suited for purification from crude preparations and easily amenable to enzymatic RNA processing and high-throughput applications. For development of the method, the RNA of interest used was a mutant of the adenine riboswitch aptamer (RSA_{U65C}; FIG. 2A), because this RNA has been previously purified using standard methods (Delfosse, V. et al. (2010) supra). Several ARiBo tags (FIG. 2B) and GST/λN-fusion proteins (FIG. 3) were tested to develop an optimum protocol for affinity batch purification of RNA.

Initially, affinity batch purification was performed using the ARiBo1 tag (FIG. 2B) and the GST-λN fusion protein in which the GST was directly fused to the N terminus of the λN peptide (FIG. 3). Examination of aliquots collected at the various steps of the purification revealed that the eluted RNA was contaminated with the affinity tag and the yield was rather poor (FIG. 4A). Similar results were obtained using a GST-L-λN fusion protein (FIG. 3), in which a linker sequence was inserted between the C terminus of the GST and the N terminus of λN.

In an attempt to improve the performance of the method, the GST was attached to the C terminus of λN. Several such GST/λN-fusion proteins were tested (λN-L-GST, λN⁺-L-GST, λN⁺-L⁺-GST and 2λN⁺-GST; FIG. 3), which differ according to the N peptide sequence, the linker sequence, and the number (1 or 2) of λN⁺ peptide repeats (FIG. 3). The N-peptide sequence was either the wild-type (λN) or a high-affinity variant (λN⁺) sequence (FIG. 7A). All these fusion proteins in which GST is fused to the C terminus of the N peptide resulted in improved RNA yield and purity, as illustrated for the λN⁺-L⁺-GST fusion protein in FIG. 4B.

### Example 4

### Development of the Optimal Tethering System

To systematically evaluate the performance of the method using each GST/λN fusion protein, a quantitative analysis of each step of the procedure was performed (Table 1). The efficiency of RNA capture was evaluated from the percentage of unbound fusion RNA in the load supernatant and washes. The high percentage of unbound fusion RNA observed for the GST-λN protein (≧44%) compared to other GST/λN fusion proteins (≧8-22%) indicates a lower efficiency of RNA capture for the GST-λN protein. However, the percentage of RNA self-cleavage on the resin was very efficient for all GST/λN fusion proteins; it was only slightly lower on the resin (93-99%) than in the transcription reaction (>99%). Hence, immobilization of the RNA on the resin using any GST/λN fusion protein did not significantly affect the efficiency of self-cleavage by the glmS ribozyme. The percentage of RNA eluted with respect to the expected yield from the transcription reaction was less than 50% for the GST-λN (39±2%) and GST-L-λN (49±2%) and ranged between 54-65% for λN-L-GST, λN⁺-L-GST, λN⁺-L⁺-GST and 2λN⁺-GST. The percentage of RNA purity with respect to the main RNA contaminant in the sample (the ARiBo tag), was relatively low for GST-λN (70±1%) and GST-L-λN (56±2%), but greater than 95% for λN-L-GST, λN⁺-L-GST, λN⁺-L⁺-GST and 2λN⁺-GST. Thus, the quantitative analysis confirms that the GST-λN and GST-L-λN fusion proteins exhibit lower yield and purity as compared to fusion proteins in which the GST was fused to the C terminus of λN in affinity batch purification. Greater than 99% purity was systematically obtained using GST/λN fusion proteins engineered with the G1N2K4 λN peptide variant that binds boxB RNA with picomolar affinity. The λN⁺-L⁺-GST fusion protein offers optimum performance with an RNA yield of 64.6±0.7% and RNA purity of 99.86±0.09%.

TABLE I
Results of affinity batch purification of RSA_{U65C} using
the ARiBo1 tag and different GST/λN₁₋₂₂ fusion proteins.

| Fusion proteins | GST-λN | GST-L-λN | λN-L-GST | λN⁺-L-GST | λN⁺-L⁺-GST | 2λN⁺-GST |
|---|---|---|---|---|---|---|
| Unbound RNA (%) | ≧44 ± 4 | ≧20 ± 4 | ≧17 ± 1 | ≧22 ± 3 | ≧13 ± 1 | ≧8 ± 1 |
| Cleavage in solution (%) | 99.9 ± 0.1 | 99.8 ± 0.1 | 99.8 ± 0.1 | 99.7 ± 0.2 | 99.6 ± 0.2 | 99.6 ± 0.1 |
| Cleavage on the resin (%) | 97.8 ± 0.7 | 93 ± 3 | 96 ± 1 | 97 ± 2 | 98.7 ± 0.5 | 95 ± 2 |
| RNA eluted (%) | 39 ± 2 | 49 ± 2 | 57 ± 3 | 58 ± 2 | 64.6 ± 0.7 | 61 ± 3 |
| RNA purity estimate (%) | 70 ± 1 | 56 ± 2 | 96.2 ± 0.4 | 99.1 ± 0.3 | 99.86 ± 0.09 | 99.8 ± 0.2 |

A major advantage of this new affinity purification procedure is the high purity level that is attained with for example λN⁺-L⁺-GST. This fusion protein is likely compatible with both the high-affinity GST/GSH-Sepharose™ and λboxB/λN⁺ interactions, preventing leakage of the ARiBo tag during elution. The λN⁺-L⁺-GST protein is very stable and large quantities are easily purified (˜25 mg purified protein/L media). In order to achieve maximum yield and purity for RNA purification, a 5-fold molar excess of λN⁺-L⁺-GST with respect to RNA was used. Reducing the fusion protein:RNA ratio from 5:1 to 4:1 and 3:1 did not significantly affect the purity, but resulted in lower yields (from 65% to 54% and 43%, respectively) (Table II). With a 2:1 ratio, both the purity (97%) and yield (25%) were reduced (Table II).

TABLE II
Effect of RNA:protein ratios on yields of affinity batch purification
using the ARiBo1 tag and the λN⁺-L⁺-GST fusion protein^{a}.

| | RNA:protein ratio | | | |
|---|---|---|---|---|
| | 1:2 | 1:3 | 1:4 | 1:5 |
| Unbound RNA (%) | ≧68 ± 3 | ≧54 ± 4 | ≧33 ± 1 | ≧13 ± 1 |
| Cleavage in solution | 99.8 ± 0.1 | 99.7 ± 0.2 | 99.73 ± 0.04 | 99.6 ± 0.2 |
| (%) | | | | |
| Cleavage on the | 98.5 ± 0.6 | 98 ± 1 | 95 ± 2 | 98.7 ± 0.5 |
| resin (%) | | | | |
| RNA eluted (%) | 25 ± 5 | 43 ± 1 | 54 ± 2 | 64.6 ± 0.7 |
| RNA purity estimate | 97 ± 2 | 99.4 ± 0.2 | 99.7 ± 0.2 | 99.86 ± 0.09 |
| (%) | | | | |

^{a}The GSH-Sepharose ™ resin:protein ratio was the same for all conditions, as described in Materials and Methods (Example 1).

The GST/GSH-Sepharose™ system provides several advantages for affinity purification of RNA. Control affinity purifications in which the GST-fusion protein was omitted revealed that the RNA does not bind to the GSH-Sepharose™ resin. The GSH-Sepharose™ resin is currently one of the most affordable and versatile affinity resins available on the market and it is compatible with a wide variety of applications, from large-scale production with batch method or column chromatography to small scale and high-throughput applications with spin columns, magnetizable beads or 96-well plates. It can also be easily regenerated, and this may help reduce cost, particularly for large-scale applications.

### Example 5

### Development of the Optimal ARiBo Tag

The ARiBo1 tag was created to minimize the size of the affinity tag by incorporating the λboxB RNA in the variable apical P1 stem-loop of the glmS ribozyme (Winkler, W. C. et al., 2004, supra; Cochrane, J. C. et al., 2007, supra; Barrick, J. E. et al. (2004) Proc Natl Acad Sci USA, 101, 6421-6426). A small tag may be desirable to improve the yield of in vitro transcriptions in the presence of limiting, modified or expensive nucleotides (NTPs), for example when using isotopically-labeled NTPs for NMR studies (Nikonowicz, E. P. et al. (1992) Nucleic Acids. Res., 20, 4507-4513; Batey, R. T. et al. (1992) Nucleic Acids Res, 20, 4515-4523) or non-standard NTPs for structure-function studies (Padilla, R. and Sousa, R. (1999) Nucleic Acids Res, 27, 1561-1563). To evaluate the efficiency of the ARiBo1 tag, control affinity purifications with the λN⁺-L⁺-GST fusion protein using the ARiBo2 and ARIBo3 tags were carried out, in which either one or two boxB RNAs were respectively positioned at the 3′-end of the glmS ribozyme (FIG. 2B). The quantitative analysis indicates that the ARiBo1 tag provides similar purity (≧99% for all three tags), but slightly higher RNA yields (64.9±0.7%) relative to either the ARiBo2 (49±2.0%) or ARiBo3 tags (59±2%) (Table III). Thus, by engineering a minimal affinity tag that combines the λboxB RNA and glmS ribozyme elements at the structural level rather than in a sequential manner, a slightly more efficient affinity tag was designed.

TABLE III
Results of affinity batch purification of RSA_{U65C} using the
λN⁺-L⁺-GST fusion protein and different ARiBo tags.

| | ARiBo tags | | |
|---|---|---|---|
| | ARiBo1 | ARiBo2 | ARiBo3 |
| Unbound RNA (%) | ≧13 ± 1 | ≧23 ± 2 | ≧16 ± 3 |
| Cleavage in solution (%) | 99.6 ± 0.2 | 99.4 ± 0.3 | 99.2 ± 0.1 |
| Cleavage on the resin (%) | 98.7 ± 0.5 | 96 ± 3 | 97 ± 2 |
| RNA eluted (%) | 64.6 ± 0.7 | 49 ± 2 | 59 ± 2 |
| RNA purity estimate (%) | 99.86 ± 0.09 | 99.8 ± 0.2 | 99.87 ± 0.06 |

### Example 6

### Examination of Effects of Sequence at the 3′-End of the RNA of Interest

Previous studies on the glmS ribozyme indicate that an adenine immediately upstream of the cleavage site (N−1 position) is conserved in bacteria and that mutation to a guanine at this position leads to reduced cleavage activity (Winkler, W. C. et al., 2004, supra; Roth, A. et al., 2006, supra; Barrick, J. E. et al. (2004) supra). It was also previously shown that truncation of the Bacillus subtilis 5′-UTR to one nucleotide upstream of the cleavage site resulted in active glmS ribozyme (Winkler, W. C. et al., 2004, supra). The crystal structure of the B. anthracis glmS ribozyme bound to GlcN6P revealed that the A-1 forms two hydrogen bonds with G57 (Cochrane, J. C. et al., 2007, supra).

To further examine the effect of the sequence at the cleavage site of the glmS ribozyme, the ARiBo1-fusion RSA_{U65C} was modified such that the two-nucleotide GA linker (G₇₃A₇₄ in FIG. 2A) was replaced by GG, GC, GU, or a single A and tested for self-cleavage directly in the transcription reaction (FIG. 5). Under standard cleavage conditions (0.5 mM GlcN6P, 20 mM Tris pH 7.6 and 10 mM MgCl₂ for 15 min at 37° C.), 99% cleavage was obtained for the GA and A linkers, 84% cleavage was obtained for the GG linker, but ˜11% cleavage was obtained for the GU and GC linkers. Under these conditions, the cleavage efficiency is GA˜A>GG>GC˜GU (FIG. 5). Structurally, a guanine may be able to partially substitute for A-1, however the C and U bases are smaller and may not be able to interact with G57, possibly affecting optimal binding of GlcN6P. By slightly increasing the GlcN6P concentration (2 mM) to favor GlcN6P binding, >99% cleavage was obtained in 30 min for the GG linker. By increasing the GlcN6P concentration even more (10 mM), 98% cleavage was obtained in 2 hr for both the GU and GC linkers (FIG. 5). Complete deletion of the linker resulted in lower cleavage under these conditions (<11%).

### Example 6

### Comparison of Affinity Batch Purification and Standard Gel Purification

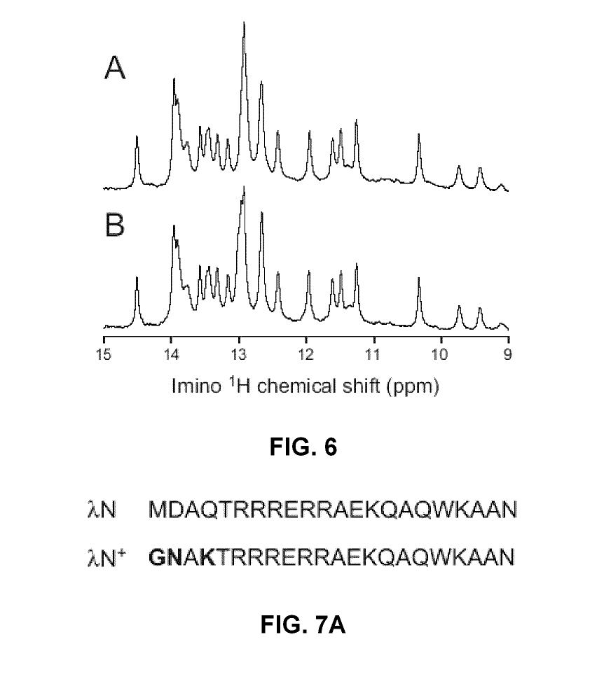

A large-scale affinity purification was performed to compare the affinity batch purification with standard purification by denaturing-gel electrophoresis. Gel purification from a 5-mL transcription reaction was completed in approximately 6 days and produced highly pure RSA_{U65C} (≧99%) at a yield of 0.58 mg/mL of transcription. To obtain an equivalent product by affinity purification with the ARiBo1-fused RSA_{U65C}, a 4-hr alkaline phosphatase step was added between the first two washes, which produces homogeneous 5′-ends. This affinity purification was completed in seven hours and produced highly pure RNA (99%) at a yield of 0.61 mg/mL of transcription. The 1D imino ¹H NMR spectrum of the affinity-purified RNA is essentially identical to that of the gel-purified RNA (FIG. 6), both being compatible with the compact three-dimensional structure of RSA_{U65C} (Delfosse, V et al., 2010, supra).

Thus, the affinity purification method described herein produces highly pure native RNA with a yield comparable to a standard denaturing gel method, but in a significantly shorter period of time. Affinity purification could be easily scaled up to a 25-50 mL transcription reaction and completed within a working day by a single individual, whereas this would require 2-3 weeks using denaturing gels.

### Example 7

### Detailed Protocol for the Production of λN⁺-L⁺-GST Fusion Protein for Affinity Immobilization of RNA

7.1 Materials

All solutions are prepared using ultrapure water, which is obtained by purifying deionized water to attain a sensitivity of at least 18 MΩcm at 21° C. All solutions are sterilized either by autoclaving or filtering (0.22 μm filter).

7.1.1 Expression of the λN⁺-L⁺-GST Fusion Protein

1. BL21(DE3) E. coli cells (Stratagene) transfected with the pET42a-λN⁺-L⁺-GST plasmid. The pET vectors use a T7 phage promoter for transcription of the cloned gene. For protein production, the recombinant plasmid is transformed into a host E. coli strain that contains a chromosomal copy of the IPTG-inducible gene for T7 RNA polymerase, such as BL21(DE3) or BL21-Gold(DE3). Store at −80° C.

2. LB Kan-50 medium: LuriaBertani (LB) broth supplemented with 50 μg/mL kanamycin just before use.

3. Isopropyl 13-D-1-thiogalactopyranoside (IPTG) solution. For 5-mL cultures: fresh 200 μL of 10 mg/mL IPTG is prepared. For the 8-L culture (fresh): 2 g of IPTG is dissolved in 24 mL water.

4. Bacterial shaking incubator with a 15-mL tube rack and 4-L flask clamps.

5. Centrifuge (Sorvall™ RC 6 Plus) with rotor (Sorvall™ SLA-3000) and 500-mL bottles.

7.1.2 Protein Purification

All solutions for protein purification are stored at 4° C.

1. Homogenization buffer: 20 mM Tris pH 7.4, 0.2 mM EDTA pH 8.0, 1 M NaCl and 1 mM DTT. The 1 mM DTT should be added just before use. For cell lysis, 150 mg protease inhibitor cocktail (Sigma-Aldrich, catalog number P8465) is also added to 80 mL of Homogenization buffer by first dissolving in 500 μl of DMSO.

2. Ultra Turrax™ T25 Basic cell disrupter (IKA) and cold metal beaker.

3. French Press with pressure cell.

4. Sonicator (Branson™ sonifier 450) with standard disruptor horn.

5. Ultracentrifuge (Sorvall™ Discovery 100SE) with rotor (Sorvall™ T-1250) and ultracentrifuge tubes.

6. GSH-Sepharose™ 4B (GE Healthcare).

7. Rotator (Thermo Scientific Labquake shaker rotisserie).

8. Centrifuge with swinging bucket rotor (IEC Centra CL2 with 215 economy swinging bucket rotor, Thermo Scientific).

9. Sintered glass Bchner funnel with 40-60 microns pore size (Pyrex).

10. Homogenization buffer with 2 M Urea: 12 g of urea is added directly to 100 mL of Homogenization buffer.

11. Phosphate buffer saline (PBS): 10 mM Na₂HPO₄, 2 mM KH₂PO₄, 2.7 mM KCl, 140 mM NaCl and pH 7.4.

12. For PBS with 20 mM reduced L-glutathione, prepare just before use by adding 0.61 g of reduced L-glutathione (Sigma-Aldrich, catalog number G4251) to 100 mL of PBS and adjusting pH to 8.0 with NaOH. Adjusting the pH of the L-glutathione solution to 8.0 maximizes the elution efficiency. Addition of L-glutathione at high concentration lowers the pH of the buffer.

13. A 0.22 μm filter unit (Millipore Steriflip™ filter unit).

14. Dialysis tubing of 29 mm diameter and 12-14 kDa MWCO with closures (Spectra/Por).

15. Magnetic stir bar and plate.

16. FPLC-A buffer: 20 mM phosphate pH 7.4, 1 mM EDTA and 1 mM fresh DTT.

17. FPLC-B buffer: FPLC-A buffer with 2 M NaCl.

18. SP-Sepharose™ High Performance column (GE Healthcare) and FPLC system. The column is constructed by packing 75 mL of SP Sepharose™ High Performance resin (GE Healthcare catalog number 17-1087-01) into an empty XK-26/20 column (GE Healthcare catalog number 18-1000-72). Store at 4° C.

19. Storage buffer: 50 mM HEPES pH 8.0, 100 mM NaCl, 2 mM fresh DTT and 20% glycerol.

20. UV/Vis spectrophotometer (Varian™ Cary-50) with a quartz cuvette.

7.1.3 Monitoring the Protein Induction and Purification by SDS Polyacrylamide Gels

1. Laemmli sample buffer (2×): Mix 1.2 mL 0.5 M Tris pH 6.8, 1.9 mL glycerol, 1 mL SDS 20%, 0.5 mL β-mercaptoethanol and a pinch of bromophenol blue. Complete to 15 mL final volume with water. Store at −20° C.

2. Tabletop microcentrifuge with rotor (Sorvall™ Pico with 24-place rotor).

3. Mini-PROTEAN™ 3 Cell Bio-Rad system.

4. 15% sodium dodecyl sulfate (SDS) polyacrylamide gels for electrophoresis. The gels could be purchased (Bio-Rad Ready™ Gel Tris-HCl gels) or prepared according to Bio-Rad's protocol.

5. Tris-glycine running buffer: 0.024 M Tris-Base, 0.192 M glycine and 0.1% SDS. Prepare first a 10× buffer solution without the SDS. Dilute 100 mL of 10× buffer into 890 mL of water and then add 10 mL of 10% SDS.

6. Molecular weight marker (Fermentas PageRuler™ Plus prestained protein ladder, catalog number SM1811) stored at −20° C.

7. Low voltage power supply (Thermo EC105).

8. Coomassie staining solution: 45% methanol, 10% acetic acid and 0.25% Brillant Blue G-250 (Fisher Scientific) in water.

9. Destaining solution: 10% methanol and 10% acetic acid in water.

7.1.4 Quality Control

1. Water bath (Isotemp™ 205, Fisher Scientific).

2. RNA sample (˜150 pmol) stored at −20° C. Here, the terminal loop of the precursor let-7g miRNA (TL-let-7g RNA) was used. For the RNase contamination assay, 1 μg of TL-let-7g, a 46-nucleotide RNA derived from the terminal loop of the let-7g precursor miRNA (5′-GCA GAU UGA GGG UCU AUG AUA CCA CCC GGU ACA GGA GAU AUC UGC A-3′, SEQ ID NO:9), was used. It is important to select either the RNA to be purified by affinity (RNA of interest) and/or an RNA, like TL-let-7g, which contains single-stranded regions (internal loops and bulges) that are susceptible to RNase cleavage (Piskounova, E. et al. (2008). J Biol Chem 283, 21310-21314).

3. Equilibration buffer: 50 mM HEPES pH 7.5. Prepare as a 10× solution.

4. Proteinase K, recombinant, PCR Grade 50 U/mL (Roche) stored at 4° C.

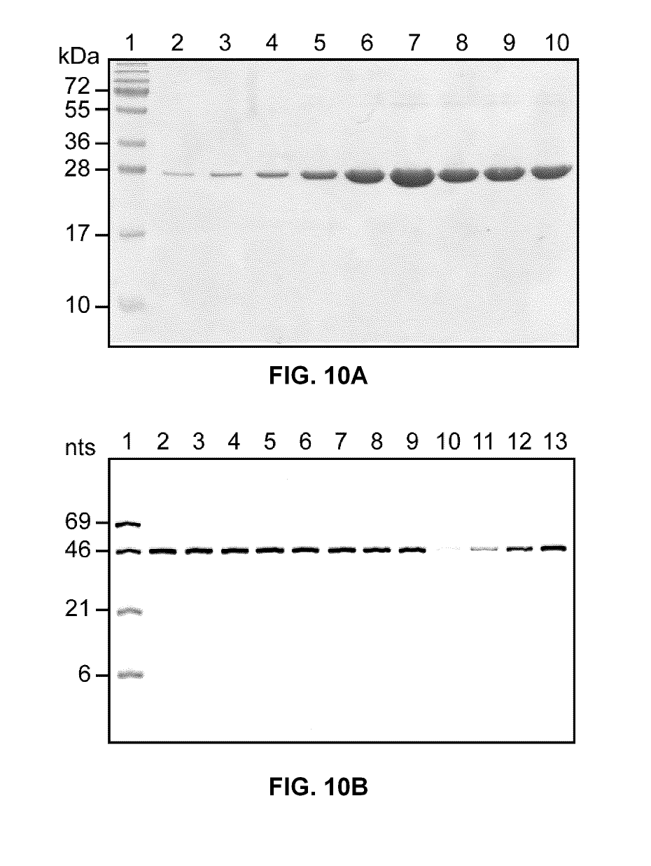

5. Gel loading buffer: 0.02 g bromophenol blue, 5 mL EDTA 0.5 M pH 8.0 and 95 mL formamide. The bromophenol blue dye is used to follow the RNA migration on the gel. RNA molecular weight markers can also be prepared using any known RNA, e.g., RNAs available in the laboratory (FIG. 10B).

6. TBE buffer: 50 mM Tris-Base, 50 mM boric acid and 1 mM EDTA. Prepare as a 10× solution.

7. 20% gel solution: 20% acrylamide:bisacrylamide (19:1), 7 M urea and TBE buffer. Store at 4° C.

8. 20% analytical denaturing polyacrylamide gel: mix 40 mL of gel solution with 200 μL ammonium persulfate 10% (w/v) and 40 μL TEMED. Immediately pour in a glass plate assembly using 20×20 cm glass plates and 0.7 mm thick comb and spacers.

9. High-voltage power supply (Thermo EC600-90).

10. SYBR™ Gold staining solution: Make a fresh 1:10,000 dilution of SYBR™ Gold nucleic acid gel stain (Invitrogen) in TBE buffer.

11. Molecular Imager FX densitometer and ImageLab™ software version 3.0 (Bio-Rad).

7.2 Methods

All procedures are carried out at room temperature unless specified otherwise.

7.2.1 Expression of the λN⁺-L⁺-GST Fusion Protein

(A) Small-Scale Induction Test.

If the pET42a-λN⁺-L⁺-GST plasmid is a new clone, it is recommended to send the plasmid for sequencing and perform the small-scale induction test to insure that overexpression of the correct fusion protein is achieved with this clone. There is no need to perform this small-scale induction test on a routine basis

1. At the end of the day, inoculate 5 mL of LB Kan-50 medium with 25 μL of a glycerol stock of the pET42a-λN⁺-L⁺-GST plasmid cloned into BL21(DE3). Let it grow overnight at 37° C. with shaking. Vigorous shaking is preferred for bacterial cell cultures; for example, 240 rpm for small cultures and 200-220 rpm for cultures in 4-L flasks. Slightly less vigorous shaking is used for 4-L flasks to prevent flasks from breaking.

2. In the morning, dilute the culture by mixing 1 mL of culture with 3 mL LB Kan-50 medium.

3. Collect a 200-4 pre-induction aliquot of the culture.

4. Induce protein expression by adding 100 μL of IPTG (10 mg/mL).

5. Incubate 4 h at 30° C. with shaking.

6. Collect a post-induction 200-4 aliquot of the culture.

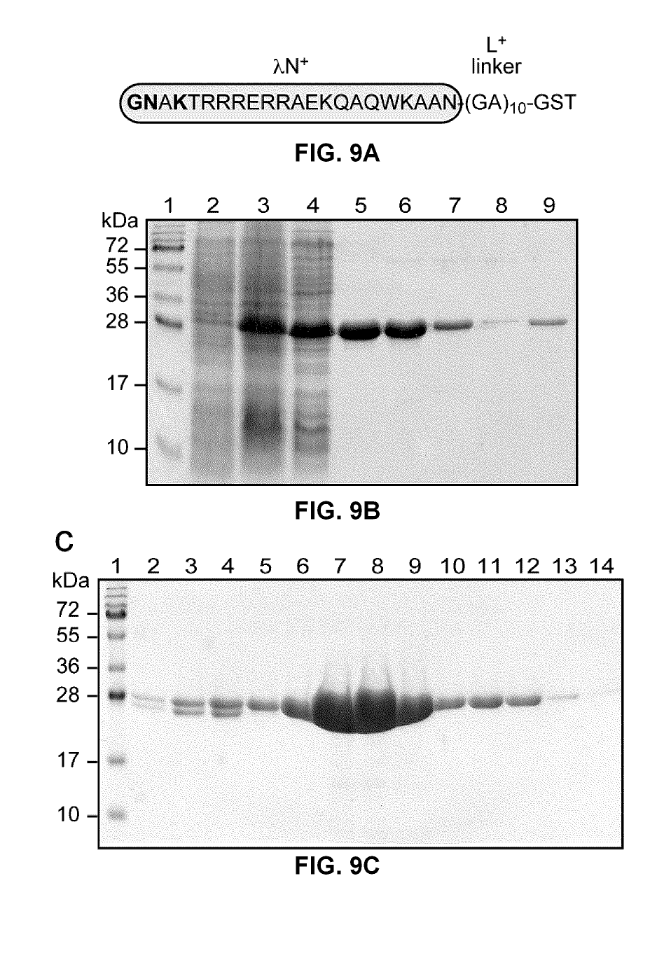

7. Verify for efficient induction on a 15% SDS polyacrylamide gel (see Section 7.1.3 above). Efficient induction of the λN⁺-L⁺-GST fusion protein is apparent from the increased intensity of the 30-kDa band in the post-induction aliquot lane (FIG. 9B, lane 3).

(B) Large-Scale Expression.

1. In the morning, inoculate 5 mL of LB Kan-50 medium with 25 μL of a glycerol stock of the pET42a-λN⁺-L⁺-GST plasmid cloned into BL21(DE3). Grow 6-8 h at 37° C. with shaking.

2. Use 1 mL of the small culture to inoculate 1 L of LB Kan-50 medium in a 4-L flask. Repeat to prepare a total of 2 L of culture. Grow overnight at 37° C. with shaking.

3. Dilute the cultures in the morning by mixing each 1-L culture with 3 L of LB Kan-50 medium and distributing equally in three 4-L flasks (1,333 mL of culture per flask). Grow for 15 min at 30° C. with shaking.

4. Collect a 500-4 pre-induction aliquot of the culture.

5. Induce protein expression by adding to each flask 4 mL of IPTG (2 g/24 mL) and grow 4 h at 30° C. with shaking.

6. Collect a post-induction 500-4 aliquot of the culture.

7. Pellet the cells in six 500-mL bottles by centrifugation at 6,000 g for 10 min and discard the supernatant. Store pellets at −80° C. until purification.

8. Verify for efficient induction on a 15% SDS polyacrylamide gel (see Section 7.1.3 above). Efficient induction of the λN⁺-L⁺-GST fusion protein is apparent from the increased intensity of the 30-kDa band in the post-induction aliquot lane (FIG. 9B, lane 3)

7.2.2 Protein Purification

For protein purification, best results are generally obtained by keeping the overall purification time as short as possible. In the following steps, all solutions are stored at 4° C. and protein-containing samples are kept on ice. If possible, the FPLC purification is conducted in the cold room. Starting at step 10, RNase free methods should be employed. It is suggested to save all fractions considered to be “waste” (ultracentrifugation pellet, GSH-Sepharose™ resin, column flow-throughs, etc.) at 4° C. until the purification is successfully completed. This is just in case a purification step is not properly carried out. The protein could be recovered from the saved fraction and the purification continued from this step.

1. Prepare 80 mL of Homogenization buffer with 150 mg of protease inhibitor cocktail.

2. Resuspend the bacterial culture pellets from an 8-L preparation (6 pellets) into the 80 mL of Homogenization buffer.

3. Homogenize the cells with an Ultra Turrax™ until the solution is homogeneous.

4. Lyse cells using a French press and a sonicator as follows. First wash the pressure cell by passing a solution of 50:50 water:ethanol through the French Press and following with two passes of water. After the washing steps, pass the cell slurry through the French Press at 800-1,000 psi and collect lysate on ice. Sonicate 10 seconds with output control set to 6 and duty cycle set to constant. Pass the cell slurry through the French Press a second time. The cell lysate should become clear and take on a darker color.

5. Transfer the cell lysate to the ultracentrifuge tubes and centrifuge for 60 min at 138,000 g and 4° C. to pellet unbroken cells and insoluble material. When the spin is completed, take a 30-μL aliquot of the supernatant.

6. During the centrifugation, prepare the GSH-Sepharose™ resin as follows. Resuspend the GSH-Sepharose™ resin in the supplier bottle by vigorous mixing. Transfer 12.5 mL of GSH-Sepharose™ slurry to a 50-mL screw-cap conical tube and add 37.5 mL of water. Centrifuge 3 min at 1,150 g in a swinging bucket and decant supernatant (when washing large amounts of resin, the supernatant can be filtered using a sintered glass Büchner funnel to recover resins lost when decanting the supernatants). Then, wash the resin twice as follows: resuspend in Homogenization buffer, centrifuge for 3 min at 1,150 g and decant supernatant. All resin washes and elutions are done using a total volume of 50 mL (buffer and resin), except for the third elution where the total volume is 25 mL.

7. Add supernatant from the high-speed spin of cell lysate to the washed GSH-Sepharose™ resin, and transfer all supernatant and resin to a 250-mL plastic bottle. Rinse the 50-mL conical tube containing the resin with a small amount (˜5 mL) of Homogenization buffer and transfer to 250-mL bottle to recover all the resin.

8. Incubate for 1 h on the rotator at 4° C.

9. After incubation, transfer the GSH-Sepharose™ resin with cell lysate back to a 50-mL screw cap conical tube, 50 mL at a time. After each addition, centrifuge the resin 3 min at 1,150 g and decant supernatant. Repeat until all the resin and lysate is removed from the 250-mL tube. Save all decanted supernatants.

10. Wash the resin twice with Homogenization buffer supplemented with 2 M Urea (urea is used to remove any bound nucleic acid. Do not use a concentration higher than 3.5 M as this is known to denature the GST protein) and twice with PBS by resuspension, centrifugation for 3 min at 1,150 g and decantation of the supernatant.

11. Elute the λN⁺-L⁺-GST fusion protein as follows. Resuspend in PBS with 20 mM reduced glutathione pH 8.0. Incubate on the rotator for 15 min at room temperature. Centrifuge the resin for 3 min at 1,150 g and decant supernatant. Take a 30-4 aliquot of the first elution supernatant. Repeat twice the elution by resuspension in PBS with 20 mM glutahione, centrifugation and decantation. Take 30-4 aliquots of the second and third elution supernatants. Pool the elution supernatants (˜100 mL) and filter through a 0.22 μm filter.

12. Resuspend the resin in PBS and take a 30-4 aliquot. Centrifuge 3 min at 1,150 g and take a 30-4 aliquot of the supernatant.

13. Transfer the pooled elution supernatant to the dialysis tubing (MWCO of 12-14 kDa) and dialyze against 4 L of FPLC-A buffer overnight at 4° C. with slow stirring.

14. Monitor the affinity batch purification on GSH-Sepharose™ resin using a 15% SDS polyacrylamide gel (see Section 7.3.1 above and FIG. 9B).

15. The following day, carefully remove the sample from the dialysis tubing with a 10-mL serological pipette and transfer to a 250-mL flask.

16. Prepare the SP-Sepharose™ column by washing 25 min with 100% FPLC-A buffer at 3 mL/min.

17. Load sample on the column through the FPLC pumps or the superloops at 3 mL/min.

18. Elute using a gradient of 0 to 100% FPLC-B buffer over 625 mL at 3 mL/min with UV detection at 280 nm. Collect 9-mL fractions. After protein purification, wash the SP-Sepharose™column for an additional 20 min with FPLC-B buffer. For long-term storage, wash the column for 20 min with a 20:80 ethanol:water solution.

19. Run a 15% SDS polyacrylamide gel to select the fractions containing the purified protein (see Section 7.3.1 and FIG. 9C).

20. Pool selected fractions (usually 8 fractions), transfer to dialysis tubing (MWCO of 12-14 kDa) and dialyze against 2 L of Storage buffer overnight at 4° C. with slow stirring.

21. The following day, carefully transfer the dialyzed sample with a 10-mL serological pipette to a 50-mL screw cap conical tube. Determine the sample volume.

22. Determine the protein concentration by UV spectroscopy at 280 nm using an extinction coefficient of 48,610 cm⁻¹ M⁻¹ (Gill, S. C. and von Hippel, P. H. (1989), Anal Biochem 182, 319-326). Yields of 200-300 mg purified protein at a concentration of 5-7 mg/mL are typically obtained.

23. Distribute in 1-10 mL aliquots and store at −20° C.

7.2.3 Monitoring the Protein Induction and Purification by SDS Polyacrylamide Gels

1. Prepare samples to be loaded on the gel. For cell culture aliquots, pellet the aliquots by centrifugation at 16,000 g for 1 min, discard the supernatant, resuspend pellet with 50 μL of water and then add 50 μL of 2× Laemmli sample buffer. For protein aliquots, add 30 μL of 2× Laemmli sample buffer directly to the 30 μL aliquots. Heat samples at 95° C. for 3 min and spin down prior to loading. Protein samples can be stored at −20° C. in Laemmli sample buffer if needed. They should be heated just prior to loading on the gel.

2. Load samples to be analyzed on an analytical 15% SDS polyacrylamide gel. To verify the induction of the small-scale induction test, load 15-μL of the pre-induction and post-induction samples (see Section 7.2.1 (A)). Load 7-μL samples to verify the induction of the large-scale culture (see Section 7.2.1(B)), monitor the affinity batch purification on GSH-Sepharose™ (see Section 7.2.2) and examine fractions of the SP-Sepharose™ column purification (see Section 7.2.2). For all applications, load at least one lane with a molecular weight marker. Run the gel at 150 V for 1.25 h.

3. Stain the gel with Coomassie staining solution for 10 min. Destain the gel with Destaining solution for 30 min to overnight.

7.2.4 Quality Control

Four simple tests are performed to verify that the λN+-L+-GST fusion protein is of sufficient quality for affinity purification of RNA.

1. To evaluate the final protein purity, analyze 0.25, 0.5, 1.0, 2.0, 5.0 and 10 μg of purified protein on a 15% SDS polyacrylamide gel (see Section 7.2.3 and FIG. 10A). High-purity (≧97.5%) is assessed by comparing the intensity of possible contaminants in the 10 μg lane with that of the 30-kDa band in the lanes containing small amounts of purified proteins.

2. To evaluate protein stability, 5.0 μg of protein is incubated in Storage buffer at 37° C. for 0, 1, 2 and 4 h, and the protein stability is assessed on the same 15% SDS polyacrylamide gel used to evaluate protein purity (see FIG. 10A). High stability (no visible degradation ≧5%) is determined by comparing the intensity of bands from degradation products, if detectable, with the intensity of the 30-kDa band in the lanes containing small amounts of purified proteins.

3. To ensure that the purified protein has the expected molecular weight (29 647 Da), a protein sample is submitted to LC-MS analysis. For example, 200 μL of a 1 mg/mL sample (diluted in water) is sent to a Mass Spectrometry facility (Regional Center for Mass Spectrometry, Department of Chemistry, Universite de Montreal) for LC-MS analysis.

4. To ensure that the protein sample is RNase free, incubate 70 pmol of an RNA [here TL-let-7g] with 350 pmol of the purified protein (10.4 μg λN⁺-L⁺-GST) in 1× Equilibration buffer (8 μL final volume) for 0, 1, 2 and 4 h at 37° C. Perform the same incubation replacing the purified protein by the volume equivalent of Storage buffer. Once the incubations are completed, add 0.05 U proteinase K to the protein-containing samples and leave at 37° C. for an additional 15 min. Analyze samples on an analytical 20% denaturing polyacrylamide gel stained with SYBR™ Gold. To prepare samples to be loaded on the gel, dilute the RNase test samples 20 fold with water and mix the volume of diluted sample corresponding to 50 ng RNA (7.55 μL for TL-let-7g RNA) with 10 μL of gel loading buffer. Also prepare control samples containing various amounts of purified RNA (2.5, 10, 25 and 50 ng RNA in sample volume ≦8 μL) by adding 10 μL of gel loading buffer. Load samples to be analyzed on a 20% analytical denaturing polyacrylamide gel pre-ran in TBE buffer at 600 V for 30 min. Run the gel in TBE buffer at 600 V for about 2 h, until the bromophenol blue is 2-4 cm from the bottom of the gel. Stain the gel with SYBR™ Gold staining solution for 10 min, scan on a Molecular Imager and quantify band intensities. No visible RNA degradation (≦5%) is assessed by comparing the intensity of possible contaminants with the intensity of the RNA band of the control samples (see FIG. 10B)

### Example 8

### Detailed Protocol for Affinity Batch Purification of RNAs Using ARiBo Tags

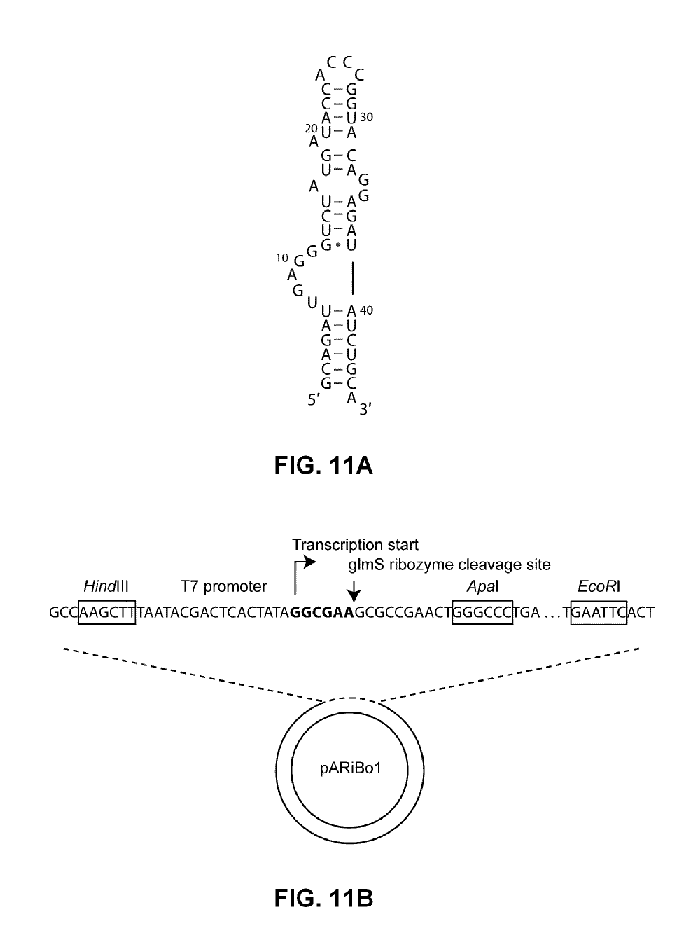

Described below are the experimental details for affinity purification of an RNA of interest using an ARiBo tag, including the cloning and preparation of the plasmid template, in vitro transcription, glmS ribozyme cleavage optimization, small-scale (3.5 nmol) and large-scale (0.25 affinity batch purifications and quantitative analysis of the purification from denaturing gels stained with SYBR™ Gold. This procedure was originally developed for purification of a stable purine riboswitch aptamer mutant (RSA_{U65C}), as described above, and it is applied below for the purification of the terminal loop of the let-7g precursor miRNA (TL-let-7g; FIG. 11A), an important target of the pluripotency factor Lin28 (Piskounova, E. et al. (2008) J Biol Chem 283, 21310-21314).

8.1 Materials

All solutions are prepared using ultrapure water, which is obtained by purifying deionized water to attain a sensitivity of at least 18 MΩcm at 21° C. All solutions are sterilized either by autoclaving or filtering (0.22 μm filter).

8.1.1 Preparation of Plasmid for Transcription of ARiBo-Fusion RNAs

1. The pARiBo1 plasmid described above stored at 4° C.

2. Forward and reverse oligonucleotide primers for mutagenesis: 20 ng/μL stocks stored at 4° C. For purification of TL-let-7g, the following primers were used 5′-GCT TTA ATA CGA CTC ACT ATA GCA GAT TGA GGG TCT ATG ATA CCA CCC GGT ACA GGA GAT ATC TGC AGC GCC GAA CTG GGC C-3′ (TL-let-7g-fwd, SEQ ID NO:66) and 5′-GGC CCA GTT CGG CGC TGC AGA TAT CTC CTG TAC CGG GTG GTA TCA TAG ACC CTC AAT CTG CTA TAG TGA GTC GTA TTA AAG C-3′ (TL-let-7g-rev, SEQ ID NO:67).

3. PfuUltra High-Fidelity™ DNA polymerase 2.5 supplied with 10×PfuUltra™ reaction buffer (Agilent Technologies). Store at −20° C.

4. 10 mM dNTP mixture: prepare by combining 1/10 dilution of 100 mM dATP, dTTP, dCTP, dGTP stocks (Invitrogen) in water. Store at −20° C.

5. Dimethyl sulfoxide (DMSO) for molecular biology ≧99.9% (Sigma-Aldrich).

6. Thermal cycler (Eppendorf Mastercycler™ gradient).

7. DpnI 20,000 U/mL restriction enzyme (New England Biolabs, catalog number R0176L) stored at −20° C.

8. Subcloning efficiency DH5α chemically competent E. coli (Invitrogen) stored at −80° C.

9. LB-Amp plates and media: LuriaBertani (LB) plates and media supplemented with 100 μg/mL ampicilin just prior to use.

10. Bacterial plate incubator (Fisher Scientific Isotemp Compact Incubator).

11. Bacterial shaking incubator with a 15-mL tube rack, 500-mL flask clamps and 4-L flask clamps.

12. 50% glycerol.

13. DNA mini-prep kit (AxyPrep™ Plasmid miniprep kit from Axygen biosciences).

14. Sequencing primer for pARiBo1: 5′-TCA CAC AGG AAA CAG CTA TGA CCA-3′ (SEQ ID NO:68). Prepare as a 5 pmol/μL stock and store at 4° C.

15. Qiagen plasmid kits: Qiafilter™ Plasmid Maxi Kit and/or Qiafilter™ Plasmid Giga Kit.

16. Centrifuge (Sorvall™ RC 6 Plus) with rotor (Sorvall™ SLA-3000) and 500 mL bottles.

17. EcoRI 100,000 U/mL restriction enzyme (New England Biolabs, catalog number R0101M) stored at −20° C.

18. EcoRI/HEPES buffer (10×): 0.5 M HEPES pH 7.5, 0.1 M MgCl₂, 1 M NaCl, 0.2% Triton X-100 and 1 mg/mL BSA. Store at −20° C. Although transcription reactions are generally performed in a Tris-HCl buffer, Tris is known to activate the glmS ribozyme and could lead to significant amount of glmS cleavage in the transcription reaction. Tris buffers should therefore preferably be avoided in the transcription reaction and in buffers used for components of the reaction such as the DNA template, the RNAsin Ribonuclease inhibitor dilution and the T7 RNA polymerase. Tris buffers should also preferably be avoided in the first steps of affinity purification.

8.1.2 In Vitro Transcription of ARiBo-Fusion RNAs and Cleavage Optimization

1. 400 mM HEPES pH 8.0.

2. 1 M DTT prepared fresh.

3. 1% Triton™ X-100.

4. 25 mM spermidine stored at −20° C.

5. Nucleotides solutions: 100 mM ATP, 100 mM CTP, 100 mM GTP, 100 mM UTP and 100 mM GMP (all from Sigma-Aldrich). All NTP solutions are prepared on ice and adjusted to pH 8.0 using NaOH. Store at −20° C.

6. Transcription buffer: 40 mM HEPES pH 8.0, 50 mM DTT, 0.1% Triton X-100, 1 mM spermidine, 4 mM ATP, 4 mM CTP, 4 mM GTP and 4 mM UTP. Prepare just before use.

7. 0.5 M MgCl₂.

8. RNAsin™ Ribonuclease inhibitor 40 U/μL (Promega). Prepare a 1:120 dilution (0.3 U/μL) in RNAsin™ buffer (20 mM HEPES pH 7.6, 50 mM KCl, 8 mM DTT and 50% v/v glycerol) and add 1 μL of the dilution to a 100 μL transcription. Store at −20° C.

9. Linearized plasmid DNA template (˜1.5 mg/mL). Store at 4° C.

10. His-tagged T7 RNA polymerase (˜6 mg/mL); made in-house and stored at −20° C.

11. Optional: yeast inorganic pyrophosphatase 2,000 U/mL (New England Biolabs, catalog number M0296S). Store at −20° C. A white precipitate generally forms during the course of the transcription reaction, which results from the formation of an insoluble complex between Mg²⁺ and pyrophosphate. A large amount of precipitate is often associated with good transcription yields. Inorganic phosphatase can be added to the reaction to reduce the precipitate and can sometimes increase the yield of transcription.

12. Optional: glucose-6-phosphate (Sigma-Aldrich). Prepare on ice as a 40 mM stock and adjust to pH 8.0 using NaOH. Aliquot and store at −20° C. GlmS ribozyme self-cleavage may be observed in transcription reaction if activators of the ribozyme are present in the reaction. Glucose-6-phosphate is an inhibitor of the glmS ribozyme that can be used to reduce ARiBo-tag cleavage without significantly affecting the transcription yield and subsequent GlcN6P-induced cleavage.

13. 0.5 M EDTA pH 8.0.

14. 0.5 M Tris pH 7.6.

15. 40 mM GlcN6P: prepare on ice and adjust to pH 7.6 using NaOH. Aliquot and store at −20° C.

16. Purified RNA control (100 ng) stored at −20° C. For the purified RNA control, it is preferable to use the same RNA as the one being purified. If this RNA is not available, a control purified RNA of similar size can be used to estimate the expected yield of the RNA of interest.

8.1.3 Affinity Batch Purification

1. In-house purified λN⁺-L⁺-GST fusion protein [5-7 mg/mL, see above] stored at −20° C.

2. Equilibration buffer: 50 mM HEPES, pH 7.5. Adjust pH with NaOH.

3. Tube rotator (Thermo Scientific Labquake™ shaker rotisserie)

4. Spin cups (Pierce, catalog number 69702).

5. GSH-Sepharose™ 4B (GE Healthcare) stored at 4° C.

6. Tabletop microcentrifuge with rotor (Sorvall™ Pico with 24-place rotor).

7. Phosphate buffer saline (PBS): 10 mM Na₂HPO₄, 2 mM KH₂PO₄, 2.7 mM KCl, 140 mM NaCl and pH 7.4.

8. Modified CIP buffer: 50 mM HEPES pH 8.5 and 0.1 mM EDTA.

9. Calf intestinal alkaline phosphatase 1 U/μL (Roche, catalog number 10713023001) stored at 4° C.

10. Elution buffer: 20 mM Tris pH 7.6, 10 mM MgCl₂ and 1 mM GlcN6P (or the concentration of GlcN6P determined by the cleavage assay). Prepare fresh before use from stock solutions (see 8.1.2).

11. 2.5 M NaCl.

12. PBS with 20 mM reduced L-glutathione: prepare just before use by adding 0.61 g of reduced L-glutathione (Sigma-Aldrich, catalog number G4251) to 100 mL of PBS and adjust pH to 8.0 with NaOH. It is important to adjust the pH of the L-glutathione solution to 8.0 in order to maximize the elution efficiency of the GST-fusion protein. Addition of L-glutathione at high concentration lowers the pH of the buffer.

13. Centrifugal filter device (Amicon™ Ultra-15 centrifugal device from Millipore).

14. Centrifuge (Sorvall™ biofuge Stratos) with swinging-bucket rotor.

15. UV/Vis spectrophotometer (Varian™ Cary-50) with a quartz cuvette.

16. Steriflip™ filter unit (Sterile 50-mL disposable vacuum filtration system from Millipore, catalog number SCGP00525).

8.1.4 Quantitative Analysis of Affinity Batch Purification

All solutions used for denaturing gel electrophoresis are filtered through a 0.22 μm filter membrane to minimize detection of undesirable fluorescent speckles on the gel.

1. Gel loading buffer: 20 mg bromophenol blue, 5 mL EDTA 0.5 M pH 8.0 and 95 mL formamide.

2. TBE buffer: 50 mM Tris-Base, 50 mM boric acid and 1 mM EDTA. Prepare as a 10× stock solution.

3. 10% gel solution: 10% acrylamide:bisacrylamide (19:1), 7 M urea in TBE buffer. Store at 4° C.

4. 10% analytical denaturing polyacrylamide gel: mix 40 mL of gel solution with 200 μL ammonium persulfate 10% (w/v) and 40 μL TEMED. Immediately pour in a glass plate assembly using 20×20 cm glass plates and 0.7 mm thick comb and spacers.

5. High-voltage power supply (Thermo EC600-90).

6. SYBR™ Gold staining solution: make a fresh 1:10,000 dilution of SYBR™ Gold nucleic acid gel stain (Invitrogen) in TBE buffer.

7. Molecular Imager™ FX densitometer and ImageLab™ software version 3.0 (Bio-Rad).

8.2 Methods

All procedures are carried out at room temperature unless specified otherwise.

8.2.1 Preparation of Plasmid for Transcription of ARiBo-Fusion RNAs

(A) Cloning.

For cloning the plasmid used for transcription of small RNAs (<50 nucleotides), it is straightforward to use the modified QuikChange™ II site-directed mutagenesis procedure (Agilent Technologies), as described here for TL-let-7g (see FIG. 11A). For longer RNA sequences, other cloning procedures can be used. For example, a double-stranded DNA template insert (annealed synthetic oligonucleotides or PCR fragment) digested with HindIII and ApaI can be ligated within the pARiBo1 plasmid digested with the same two restriction enzymes and dephosphorylated (see FIG. 11B).

1. Design forward and reverse oligonucleotide primers for mutagenesis.

2. Prepare a PCR amplification reaction (50 μL) that includes 5 μL 10×PfuUltra™ reaction buffer, 100 ng of pARiBo1 supercoiled plasmid DNA, 125 ng of forward primer, 125 ng of reverse primer, 1 μL of 10 mM dNTP mixture, 3 μL DMSO, water to complete to 49 μL and 1 of PfuUltra™ High-fidelity DNA polymerase.

3. Run the PCR reaction in the thermal cycler as follows: 1) 95° C. 2 min to start; 2) then 18 cycles of 95° C. 1 min, 60° C. 1 min, 68° C. 7 min; and 3) finish with 68° C. 7 min and 4° C. 5 min.

4. Add 30 U of Dpn1 to the PCR mixture and incubate at 37° C. for 2 h in order to digest the parental DNA.

5. Transform 3 μL of the Dpn1-digested PCR mixture into 50 μL of DH5α competent cells, spread the transformation on the surface of a LB-Amp plate and incubate overnight at 37° C. Store the plate at 4° C.

6. At the end of the next day, inoculate 5 mL of LB-Amp medium with an individual colony. Repeat for two other small cultures. Incubate overnight at 37° C. with shaking. Vigorous shaking is preferred for bacterial cell cultures; for example, 240 rpm for small cultures (5 mL and 150 mL) and 200-220 rpm for cultures in 4-L flasks. Slightly less vigorous shaking is used for 4-L flasks to prevent flasks from breaking.

7. The next day, prepare glycerol stocks for each culture by mixing 600 μL of the culture with 400 μL of 50% glycerol. Store at −80° C.

8. Purify plasmids using a mini-prep kit. Perform DNA sequencing using the pARiBo1 sequencing primer and select a clone with the right sequence.

(B) Plasmid Preparation.

Plasmid preparation can be performed at different scales depending on the anticipated needs for transcription. Small bacterial cultures (150 mL) are used for plasmid purification with a Maxi-prep kit to obtain 0.3-1 mg of plasmid, which is sufficient for several small-scale transcriptions. Large bacterial cultures (2.5 L) are used for plasmid purification with a Giga-prep kit to obtain 7.5-15 mg of plasmid, which is sufficient for large-scale transcriptions.

1. In the morning, inoculate 5 mL of LB-Amp media with 25 μL of a glycerol stock of the new plasmid, here pTL-let-7g-ARiBo1, cloned into DH5a. Grow 6-8 hours at 37° C. with shaking.

2. For Maxi preps, use 0.3 mL of the small culture to inoculate 150 mL LB-Amp in a 500 mL flask. For Giga preps, use 1 mL of the small culture to inoculate 1.25 L of LB-Amp media in a 4-L flask and repeat to prepare a total of 2.5 L of culture. Grow overnight at 37° C. with shaking.

3. Pellet the cells by centrifugation at 6,000 g for 10 min using either one 500-mL bottle (Maxi prep) or six 500-mL bottles (Giga prep). Discard the supernatant. The pellets can either be stored at −80° C. until needed or processed immediately.

4. Extract the plasmid from the cell pellet using either the Qiagen QIAfilter™ Plasmid Maxi Kit or the Qiagen QIAfilter™ Plasmid Giga Kit, according to the supplied protocol. Resuspend the purified plasmid in water. Determine the DNA concentration by UV spectroscopy (λ=260 nm, 1 OD₂₆₀=50 mg/mL double-stranded DNA).

5. Linearize the plasmid with the EcoRI restriction enzyme, keeping a small amount of undigested plasmid (˜5 μg) for controls on agarose gels. For 300 μg of plasmid, use 300 U of EcoRI, 20 μL of 10× EcoRI/HEPES buffer and complete volume to 200 μL with water. Scale up this reaction as needed for larger quantities of plasmid. Incubate overnight at 37° C.

6. Verify that the plasmid is completely linearized on a 1% agarose gel. In order to maximize the yield of transcription, restriction enzyme digestion should ideally be performed to completion. Uncut plasmids allow efficient continuous transcription, giving rise to very long transcripts that use up the NTPs.

7. Once the plasmid is completely cut, inactivate the restriction enzyme by heating at 65° C. for 5 min and transferring on ice. The linearized DNA plasmid is stored at 4° C. as is.

8.2.2 In Vitro Transcription of ARiBo-Fusion RNAs and Cleavage Optimization

Several small-scale transcriptions (100 μL) are usually performed in order to optimize the yield for large-scale transcriptions (5-50 mL).

(A) Small-Scale Transcription Optimization.

1. Typically, six small transcription reactions (100 are set up. The standard reaction contains 40 mM HEPES pH 8.0, 50 mM fresh DTT, 0.1% Triton™ X-100, 1 mM spermidine, 20 mM MgCl₂, 4 mM of each NTP (ATP, UTP, GTP, CTP), 8 μg linearized plasmid DNA, 0.3 U RNAsin™ Ribonuclase inhibitor and 1 μL T7 RNA polymerase 6 mg/mL. The five other transcription reactions are as the standard reaction except that one factor is varied in each reaction: the concentration of MgCl₂ (15 mM and 25 mM instead of 20 mM), the template concentration (12 μg/100 μL instead of 8 μg/100 μL), the nucleotide concentration (4 mM GMP is added), or the enzyme concentration (2 μL instead of 1 μL T7 RNA polymerase 6 mg/mL). If needed, 0.01-0.05 U inorganic pyrophosphatase and 5-10 mM glucose-6-phosphate can be added.

2. Incubate for 3 h at 37° C.

3. Stop the transcription reaction by adding the necessary volume of 0.5 M EDTA pH 8.0 such that the EDTA concentration is equal to the MgCl₂ concentration. Store at −20° C.

4. Analyze samples (1.5 μL of a 1:200 dilution) on an analytical 10% denaturing polyacrylamide gel stained with SYBR™ Gold (see section 8.2.4).

5. Scan the gel on a Molecular Imager and quantify the intensity of the high-molecular weight band on the gel (see section 8.4). Select the transcription condition that produces the highest yield of ARiBo-fusion RNA according to the intensity of this band.

(B) Optimization of Cleavage Conditions with GlcN6P.

1. Once the small-scale transcription reactions are completed, the condition for glmS ribozyme cleavage is optimized. One can use either the transcription reaction that produces the highest yield or simply the standard transcription reaction (see section 8.2.2(A) above). Three 100-4 cleavage reactions are typically set up that contain 3 μL of the transcription reaction, 20 mM Tris pH 7.6, 10 mM MgCl₂ and varying concentrations (1, 2 and 4 mM) of GlcN6P.

2. Incubate the cleavage reactions at 37° C. Remove 5-4 aliquots from the reaction mixture at specific times (0, 15, 30 and 60 min) and mix with 95 μL of gel loading buffer to stop the cleavage reaction.

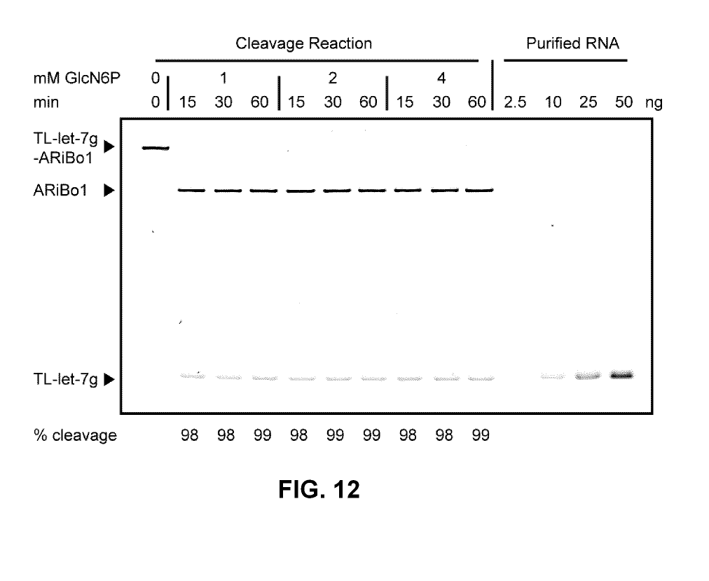

3. Analyze cleavage samples (5-20 ng of cleaved RNA per well, here 10 μL of the stopped cleavage mix) together with control samples containing various amounts of purified RNA (2.5, 10, 25 and 50 ng RNA) on an analytical 10% denaturing polyacrylamide gel stained with SYBR™ Gold (see Section 8.2.4 and FIG. 12).

4. Scan the gel on a Molecular Imager™ and quantify the intensity of the gel bands corresponding to the ARiBo-fusion RNA and the ARiBo tag (see Section 8.2.4).

5. Determine the % of cleavage in solution:

(BI_{ARiBo}/nt_{ARiBo})/{(BI_{ARiBo}/nt_{ARiBo})+(BI_{Fusion}/nt_{Fusion})}×100% (Equation 1)

where BI_{ARiBo} and BI_{Fusion} are the band intensities of the ARiBo tag and ARiBo-fusion RNA, respectively, whereas nt_{ARiBo} and nt_{Fusion} are the number of nucleotides of these RNAs.

6. Select the best cleavage condition. Although >95% cleavage is typically obtained with a 15 min incubation in 1 mM GlcN6P (see FIG. 12), efficient cleavage of some ARiBo-fusion RNA may require a higher concentration of GlcN6P and/or a longer incubation time. This is generally the case when the nucleotide at the 3′-end of the RNA of interest is not an unpaired adenine (Di Tomasso, G. et al. (2011) Nucleic Acids Res 39, e18). For RNAs with an unpaired adenine at their 3′-end, if little or no cleavage is observed after a 1-h incubation at 4 mM GlcN6P, this may indicate misfolding of the ARiBo-fusion RNA. In such case, one or more of the following conditions can be tested to help improve the cleavage yield: 1) use even longer incubation time and/or higher GlcN6P concentration; 2) increase or lower the concentration of MgCl₂; 3) refold the RNA by heating and subsequently cooling; 4) increase the cleavage temperature (e.g., 42° C.); and 5) store the transcription reaction at 4° C. (instead of −20° C.) or perform purification immediately after the transcription is completed. It is important to bear in mind that glmS ribozyme cleavage is typically performed at 37° C. with 10 mM MgCl₂; incubations at higher temperatures and for longer periods of time may cause undesirable degradation of the RNA.

7. Estimate the expected yield of RNA (in mg/mL transcription and nmol/μL transcription) for the selected cleavage condition. The data in the control lanes are used to derive a standard curve, from which is determined the quantity (in ng) of RNA (N_{RNA}) corresponding to the amount of transcription volume loaded on the gel (15 mL; see FIG. 12).

(C) Large-Scale Transcription

1. The reaction conditions for the large-scale transcription (typically 5 to 50 mL) are determined from the small-scale transcription optimization and simply scaled up according to the RNA needs. The yield of the large-scale transcription (in mg RNA/mL transcription) should be the same as for the small-scale reaction if care is taken to use the same solutions for both reactions.

2. Once the transcription is completed, the yield of the large-scale transcription is compared to that of the small-scale transcription on a 10% denaturing polyacrylamide gel stained with SYBR™ Gold (see Section 8.2.4). Samples loaded on the gel are: 1) aliquots from the small-scale and large-scale transcription reactions (1.5 μL of a 1:200 dilution of the transcription reaction); small-scale and large-scale transcription reactions after cleavage of the ARiBo tag with GlcN6P under optimized conditions (see Section 8.2.2(A) above; 3 and 64 of a 1:10 dilution of the cleavage reaction); and control samples containing various amounts of purified RNA (2.5, 10, 25 and 50 ng RNA).

3. Scan the gel with a Molecular Imager™ and quantify band intensity (see Section 8.2.4).

4. Determine the % of cleavage in solution for the small-scale and large-scale transcription reactions using Equation 1 (see above). The % of cleavage should be similar for both transcriptions.

5. Estimate the expected yield of RNA (in mg/mL transcription and nmol/μL transcription) for the small-scale and large-scale transcription reactions, as described above. The expected yield should be similar for both reactions.

8.2.3 Affinity Batch Purification

In this section, the affinity batch purification procedure is described in details for small-scale (3.5 nmol) applications, and guidelines are provided for adapting the procedure to large-scale applications (0.25 μmol). Only one main modification has been made with respect to the original protocol (Di Tomasso, G. et al. (2011) Nucleic Acids Res 39, e18). To circumvent the process of decanting the supernatant after centrifugation of GSH-Sepharose™ resin, the use of spin cups was introduced for small-scale purifications and that of Steriflip™ filter units for large-scale purifications. The use of these devices is more straightforward, and it also improves time efficiency and prevents the loss of resins during the purification. For the procedure described below, all incubations are performed at room temperature with gentle rotation using a tube rotator, unless otherwise mentioned, and all centrifugation steps are performed for 1 min at 5000 g. For all incubations, the top of the spin cup may be covered with a parafilm to prevent RNase contamination.

(A) Small-Scale Purification

1. Prepare the RNA-protein mix as follows: in a 1.5-mL eppendorf tube, add 17.5 nmol of λN⁺-L⁺-GST fusion protein to a transcription volume that corresponds to 3.5 nmol of RNA to be purified (223 μg for the TL-let-7g RNA). Adjust to a total volume of 400 μL with Equilibration buffer. Incubate 15 min. The protein:RNA ratio may need to be optimized to maximize RNA yield and purity. A 5:1 protein:RNA molar ratio is typically used to provide high RNA yield and purity. Ratios as low as 3:1 may be used without sacrificing sample purity but will likely result in slightly lower yields, whereas higher ratios (e.g. 8:1) may provide slightly higher yields but require excessive amounts of purified fusion protein (see Example 4 above).

2. In the meantime, prepare the GSH-Sepharose™ matrix in a spin cup. Spin cups are convenient tools for affinity batch purification with small volume of resin (20-400 μL), since they prevent the loss of resins and improve time efficiency. Add 125 μL of GSH-Sepharose™ resin (163 μL of the 77% slurry) to the spin cup. Wash twice by adding 400 μL of PBS and centrifuging immediately.

3. Add the RNA-protein mix to the washed resin directly in the spin cup. Incubate 15 min and centrifuge. Keep the load eluate (LE) on ice for quantitative analysis on gel.

4. Wash the RNA-loaded resin three times as follows: add 400 μL of Equilibration buffer, incubate 5 min and centrifuge. Keep the wash eluates (W1, W2 and W3) on ice for quantitative analysis. If needed, an alkaline phosphatase step can be inserted between the first and second washes. For small-scale applications, standard reaction conditions can be used: add 35 U of calf intestinal alkaline phosphatase (10 U/nmol RNA) in 400 μL modified CIP buffer, incubate at 37° C. for 30 min with periodic inversion of the spin cup, then transfer to room temperature for 5 min and centrifuge. For large-scale applications, reaction conditions can be modified to reduce enzyme cost by using 130 U/μmol RNA and incubating for 4 h at 37° C. with periodic inversion of the Steriflip™.

5. Elute the RNA as follows: add 400 μL of Elution buffer, incubate at 37° C. for 15 min (or the optimal time determined from the cleavage assay; see above) with periodic inversion of the tube, then at room temperature for 5 min and centrifuge. Keep the RNA elution sample (E1) on ice for quantitative analysis and further processing. After elution with GlcN6P, wash the resin twice as follows: add 400 μL of Equilibration buffer, incubate 5 min and centrifuge. Keep the RNA elution samples (E2 and E3) on ice for quantitative analysis and further processing. In some cases, although >95% glmS cleavage is obtained under optimized cleavage conditions, the RNA of interest may be difficult to elute because it remains bound to the resin. In these cases, RNA elution could be facilitated by adding NaCl to the RNA elution buffers, but care should be taken to minimize co-elution of the ARiBo tag. We suggest modifying the elution steps as follows: 1) after the E1 incubations, add 0.5 M NaCl and 50 mM EDTA, incubate 5 min and centrifuge; 2) after the E2 and E3 incubations, add 0.1-0.25 M NaCl, incubate 5 min and centrifuge.

6. Wash the resin to remove residual RNA as follows: add 400 μL of 2.5 M NaCl; incubate 5 min; and centrifuge. Keep the eluate (NaCl) on ice for quantitative analysis. Resuspend the resin in 125 μL PBS.

7. To completely regenerate the resin, the used resin is first collected in a 50-mL screw-cap conical tube until a significant amount of resin is available (≧5 mL). The resin is first filtered in a Steriflip™ filter unit and the Steriflip is kept for the subsequent wash steps. To use the Steriflip™ for washing resins and large-scale affinity batch purifications, proceed as follows. The Steriflip™ filter unit is first attached to the top of a 50-mL conical tube used for the incubation (tube 1), flipped over and a vacuum is applied. The bottom 50-mL conical tube containing the eluate (tube 2) is capped, kept on ice and replaced by a new 50-mL conical tube (tube 3). The Steriflip™ filter is then flipped over again, the bottom 50-mL conical tube (tube 1) is carefully detached from the filter unit, filled with the buffer used for the next incubation step, reattached to the Steriflip™ filter and gently mixed several times to ensure that all the resin is recovered from the filter surface. During the following incubation period, the filter unit on tube 1 is replaced by a screw cap and kept under RNase-free conditions for the next filtering step. The resin is then washed with 4× resin volume of solution: twice with PBS (incubate 5 min and filter), three times with 20 mM L-glutathione in PBS (incubate 15 min and filter) and 20% ethanol (incubate 5 min and filter). The resin can then be stored in 20% ethanol at 4° C.

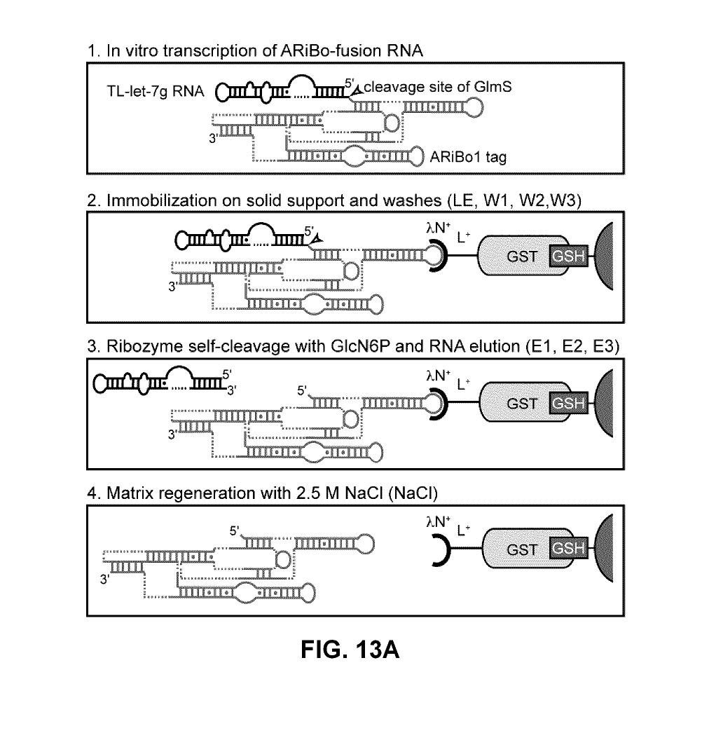

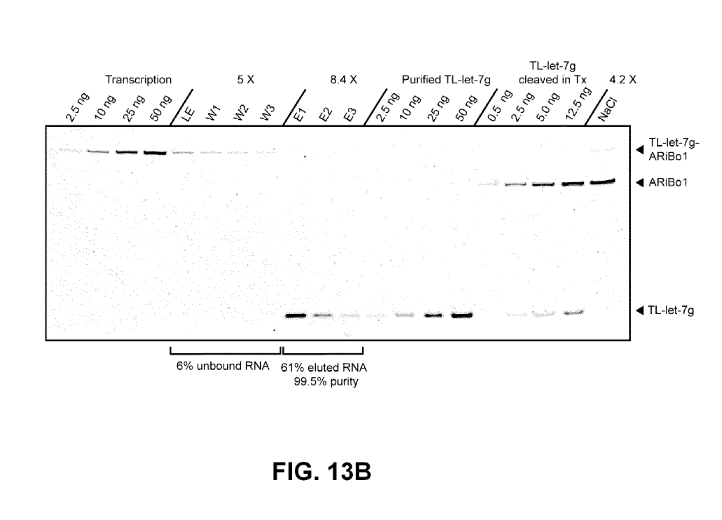

8. Once the affinity purification is completed, the purification is evaluated on a 10% denaturing polyacrylamide gel stained with SYBR™ Gold (Section 8.2.4 and FIG. 13). Samples loaded on the gel are: 1) control samples containing various volumes of the transcription reaction corresponding to specific amounts of ARiBo-fusion RNA (2.5, 10, 25 and 50 ng RNA); 2) aliquots from load and wash eluates corresponding to 250 ng of ARiBo-fusion RNA assuming that 100% of the input RNA is present in each eluate (in the case of the ARiBo-fusion TL-let-7g (a 193-nucleotide RNA of 63.7 kDa), 3.5 nmol or 223 μg would be present in the 400 μL flow through, and thus 4.49 μL of the 1:10 dilution would represent 250 ng of this RNA); 3) aliquots from the RNA elution corresponding to 100 ng of the RNA assuming a 100% purification yield at each step (In the case of the TL-let-7g RNA (a 46-nucleotide RNA of 15.2 kDa) 3.5 nmol or 53.15 μg of TL-let-7g would be present in the 400 μL RNA elution sample, and thus 7.52 μL of the 1:10 dilution would represent 100 ng of this RNA and 3.76 μL of the 1:10 dilution would represent 50 ng of this RNA); 4) control samples containing various amounts of purified RNA (2.5, 10, 25 and 50 ng RNA); 5) control samples containing various volumes of the transcription reaction cleaved with GlcN6P and corresponding to specific amounts of purified RNA (0.5, 2.5, 5.0 and 12.5 ng RNA); and 6) aliquot from the NaCl wash corresponding to 50 ng of the TL-let-7g RNA of interest assuming 100% RNA recovery at this step.

9. Combine the RNA elution samples (E1, E2 and E3), concentrate using an ultracentrifugation device and exchange in appropriate storage buffer.

10. Determine the RNA concentration by UV spectroscopy at 260 nm using a conversion factor of 1 OD₂₆₀=40 μg/mL or an extinction coefficient determined experimentally for the RNA of interest (Legault, P. (1995) Structural studies of ribozymes by heteronuclear NMR spectroscopy, University of Colorado at Boulder, Boulder; Zaug, A. (1988) Biochemistry 27, 8924-8931).

(B) Large-Scale Purification

Large-scale purifications are typically performed in 50-mL screw-cap conical tubes, using a transcription volume corresponding to 0.25 μmol of ARiBo-fusion RNA, 1.25 μmol λN⁺-L⁺-GST fusion protein, 8.9 mL of GSH-Sepharose resin (11.6 mL of 77% resin slurry) and incubation volumes of 25 mL. A single Steriflip™ filter unit is used to recover the liquid phase of resin incubations at the various purification steps. In contrast to small-scale purifications, the large-scale purification needs to be evaluated by gel electrophoresis prior to discarding any wash or elution filtrate and performing any of the resin regeneration steps, including the wash with 2.5 M NaCl. This allows one to repeat the necessary steps in cases where the yield is not as high as expected.

8.2.4 Quantitative Analysis of Affinity Batch Purification

(A) Denaturing Gel Electrophoresis

1. Prepare an analytical 10% denaturing polyacrylamide gel. Pre-run the gel in TBE buffer at 425 V for 20 min.

2. Prepare gel samples as needed in volumes ≦10 μL and add 10 μL of gel loading buffer.

3. Load samples and run the gel at 425 V for 1 h 45 min, until the bromophenol blue is 2 cm from the bottom of the gel.

4. Stain the ge110 min with 200 mL SYBR™ Gold staining solution.

5. Scan on a Molecular Imager and quantify band intensities.

(B) Quantitative Analysis

The quantitative analysis described here is used for evaluating the affinity batch purification using sample loading as described in Section 8.2.3(A). Six variables are defined for this analysis: the quantities (in ng) of purified RNA (N_{RNA}), ARiBo tag (N_{ARiBo}) and ARiBo-fusion RNA (N_{Fusion}) as well as the number of nucleotides in the purified RNA (nt_{RNA}), ARiBo tag (nt_{ARiBo}) and ARiBo-fusion RNA (nt_{Fusion}).

1. For each gel, four lanes were loaded with transcription reaction containing estimated quantities of intact ARiBo-fusion RNA (Section 8.2.2(C)). The lanes with the intact ARiBo-fusion RNA are then used to derive a standard curve relating band intensity to the quantity of ARiBo-fusion RNA (N_{Fusion}).

2. For each gel, four lanes were loaded with known quantities of the purified RNA of interest (TL-let-7g), derived from OD₂₆₀ measurements. These data are used to derive a standard curve relating band intensity with the quantity of purified RNA (N_{RNA}).

3. For each gel, four lanes were loaded with transcription reaction treated with GlcN6P and containing estimated quantities of TL-let-7g (Section 8.2.2(C)). The exact quantity of TL-let-7g (N_{RNA}) detected in these lanes is calculated using the standard curve for N_{RNA}. The exact quantity of ARiBo tag (N_{ARiBo}) detected in these same reactions is derived from:

N_{ARiBo}=(N_{RNA}/nt_{RNA})×nt_{ARiBo} (Equation 2)

These lanes with the cleaved ARiBo-fusion RNA are then used to derive a standard curve relating band intensity with the quantity of ARiBo tag (N_{ARiBo}).

4. The % of cleavage in solution is determined from a control lane in which the transcription reaction is treated with GlcN6P (FIG. 13B, lane 19) using the equation:

{(N_{ARiBo}/nt_{ARiBo})/[(N_{ARiBo}/nt_{ARiBo})+(N_{Fusion}/nt_{Fusion})]}×100% (Equation 3)

5. Equation 3 is also used to calculate the % of cleavage on the resin, although this is derived from the NaCl lane (FIG. 13B, lane 20).

6. The % of unbound RNA is calculated using:

[(ΣN_{Fusion})/I_{Fusion}]×100% (Equation 4)
where ΣN_{Fusion} represents the total amount of ARiBo-fusion RNA (ng) detected in lanes LE, W1, W2 and W3, and I_{Fusion} represents the input of the same RNA in equivalent volumes of affinity batch purification (250 ng). Since the percentage of unbound RNA is based only on the quantity of fusion RNA, it represents a minimum value of the total unbound RNA. Although, not observed for the TL-let-7g purification, slower migrating species in lanes LE, W1, W2 and W3 have been observed for purification of other RNAs.

7. The % of RNA eluted is calculated using:

[(ΣN_{RNA})/I_{RNA}]×100% (Equation 5)

where ΣN_{RNA} represents the total amount of TL-let-7g (ng) detected in lanes E1, E2 and E3, and I_{RNA} represents the calculated amount of TL-let-7g expected from 100% cleavage in equivalent volumes of transcription (100 ng).

8. The % of RNA purity is calculated from the E1 lane (FIG. 13B, lane 9) using:

[N_{RNA}/(N_{RNA}+N_{ARiBo})]×100% (Equation 6)

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A construct for immobilizing a bacteriophage boxB-comprising RNA on a solid support, said construct comprising:
a boxB RNA binding peptide;
a peptide linker linked to the C-terminus of said bacteriophage N peptide; and
an immobilizing moiety capable of binding to said solid support, wherein said immobilizing moiety is linked to said peptide linker.

2-3. (canceled)

4. The construct of claim 1, wherein said boxB RNA binding peptide is a bacteriophage N peptide.

5. The construct of claim 4, wherein said bacteriophage N peptide comprises a domain of formula I:
X¹—X²-A/S—X³—X⁴—R/K—X⁵—X⁶—X⁷—R/K—R/K—X⁸—X⁹—X¹⁰—X¹¹—X¹²—X¹³—X¹⁴ (I)
wherein
X¹ is any amino acid or is absent;
X² is A, D, T or N;
X³ is Q, R or K;
X⁴ is A, T or S;
X⁵ is Y or R
X⁶ is R, K or H;
X⁷ is E or A;
X⁸ is any amino acid;
X⁹ is any amino acid;
X¹⁰ is any amino acid;
X¹¹ is any amino acid;
X¹² is any amino acid;
X¹³ is any amino acid; and
X¹⁴ is any amino acid.

6. The construct of claim 5, wherein
X¹ is M or G
X⁸ is A or R;
X⁹ is E, K or M;
X¹⁰ is K, L or E;
X¹¹ is Q, I, A, or R;
X¹² is A or I;
X¹³ is Q, E or T; and/or
X¹⁴ is W, R or L.

7. The construct of claim 6, wherein
X¹ is G;
X² is N; and/or
X³ is K.

8. The construct of claim 7, wherein said domain is Met-Asp-Ala-Gln-Thr-Arg-Arg-Arg-Glu-Arg-Arg-Ala-Glu-Lys-Gln-Ala-Gln-Trp (SEQ ID NO:2); Gly-Asn-Ala-Lys-Thr-Arg-Arg-Arg-Glu-Arg-Arg-Ala-Glu-Lys-Gln-Ala-Gln-Trp (SEQ ID NO:3) or Gly-Asn-Ala-Lys-Thr-Arg-Arg-His-Glu-Arg-Arg-Arg-Lys-Leu-Ala-Ile-Glu-Arg (SEQ ID NO:4).

9. The construct of claim 8, wherein said bacteriophage N peptide is Gly-Asn-Ala-Lys-Thr-Arg-Arg-Arg-Glu-Arg-Arg-Ala-Glu-Lys-Gln-Ala-Gln-Trp (SEQ ID NO:3).

10. The construct of claim 1, wherein said peptide linker is a poly-glycine or poly-glycine/alanine linker.

11. The construct of claim 1, wherein said peptide linker is a 20-residue peptide linker.

12. The construct of claim 11, wherein said peptide linker consists of the sequence (Gly-Ala)₁₀.

13. The construct of claim 1, wherein said immobilizing moiety is a Glutathione S-transferase (GST) polypeptide.

14. The construct of claim 13, wherein said solid support is a Glutathione Sepharose™ bead.

15. The construct of claim 1, wherein said bacteriophage boxB is bacteriophage lambda (λ) boxB.

16. The construct of claim 1, wherein said bacteriophage boxB-comprising RNA further comprises a target RNA which is targeted for immobilization.

17. A nucleic acid encoding the construct of claim 1.

18-19. (canceled)

20. A method for immobilizing a target RNA, said method comprising:
(i)
(a) providing a bacteriophage boxB-comprising target RNA comprising a bacteriophage boxB RNA and the target RNA; and
(b) contacting the bacteriophage boxB-comprising target RNA of (a) with the construct of claim 1 bound to a solid support; or
(ii)
(a) providing a bacteriophage boxB-comprising target RNA comprising a bacteriophage boxB RNA and the target RNA;
(b) contacting the bacteriophage boxB-comprising target RNA of (a) with the construct of claim 1, thereby to obtain a complex comprising the bacteriophage boxB-comprising target RNA bound to the construct; and
(c) contacting the complex with a solid support comprising a ligand capable of binding to the immobilizing moiety.

21-27. (canceled)

28. A method for purifying a target RNA, said method comprising:
(i)
(a) providing an affinity tag-comprising target RNA comprising an affinity tag and the target RNA, wherein said affinity tag comprises a bacteriophage boxB sequence and an activatable ribozyme sequence;
(b) contacting the affinity tag-comprising target RNA of (a) with the construct of claim 1 bound to a solid support;
(c) inducing activation of said activatable ribozyme; and
(d) collecting said target RNA; or
(ii)
(a) providing an affinity tag-comprising target RNA comprising an affinity tag and the target RNA, wherein said affinity tag comprises a bacteriophage boxB sequence and an activatable ribozyme sequence;
(b) contacting the affinity tag-comprising target RNA of (a) with the construct of claim 1 thereby to obtain a complex comprising the affinity tag-comprising target RNA bound to the construct;
(c) contacting the complex with a solid support comprising a ligand capable of binding to the immobilizing moiety;
(d) inducing activation of said activatable ribozyme; and
(e) collecting said target RNA.

29-32. (canceled)

33. The method of claim 28, wherein said affinity tag is incorporated at the 3′ end of said target RNA.

34. The method of claim 28, wherein said bacteriophage boxB sequence is incorporated into the variable apical P1 stem-loop of said activatable ribozyme sequence.

35. The method of claim 28, wherein said activatable ribozyme sequence is a Glucosamine-6-phosphate activated ribozyme (glmS ribozyme) sequence.

36-54. (canceled)


SEQUENCE LISTING (75 sequences)

SEQ ID NO: 1 - protein, 18 aa, Artificial Sequence
  Synthetic peptide
XXXXXXXXXXXXXXXXXX

SEQ ID NO: 2 - protein, 18 aa, Artificial Sequence
  Synthesized
MDAQTRRRERRAEKQAQW

SEQ ID NO: 3 - protein, 18 aa, Artificial Sequence
  Synthesized
GNAKTRRRERRAEKQAQW

SEQ ID NO: 4 - protein, 18 aa, Artificial Sequence
  Synthesized
GNAKTRRHERRRKLAIER

SEQ ID NO: 5 - RNA, 69 nt, Bacillus subtilis
gcgaguauaaccucaauaauaugguuugagggugucuaccaggaaccguaaaauccugacuacucgcga

SEQ ID NO: 6 - protein, 22 aa, Bacteriophage lambda
MDAQTRRRERRAEKQAQWKAAN

SEQ ID NO: 7 - protein, 22 aa, Artificial Sequence
  Synthesized
GNAKTRRRERRAEKQAQWKAAN

SEQ ID NO: 8 - DNA, 252 bp, Artificial Sequence
  Synthesized
cgccaagccttaatacgactcactatagcgagtataacctcaataatatggtttgagggtgtctaccaggaaccgtaaaatcctgactactcgcgagcgccgaactgggccctgaagaagggctcagttgacgaggtggggtttatcgagatttcggcggatgactcccggttgttcatcacaaccgcaaacttttacttaaatcattaaggtgacttagtggacaaaggtgaaagtgtgatgaattcactg

SEQ ID NO: 9 - RNA, 46 nt, Mus musculus
gcagauugagggucuaugauaccacccgguacaggagauaucugca

SEQ ID NO: 10 - DNA, 61 bp, Artificial Sequence
  Synthesized
gccaagctttaatacgactcactataggcgaagcgccgaactgggccctgatgaattcact

SEQ ID NO: 11 - RNA, 15 nt, Bacteriophage lambda
gcccugaaaaagggc

SEQ ID NO: 12 - RNA, 15 nt, Bacteriophage lambda
gcccugaagaagggc

SEQ ID NO: 13 - RNA, 20 nt, Bacteriophage phi-21
uucaccucuaaccgggugag

SEQ ID NO: 14 - RNA, 20 nt, Bacteriophage phi-21
ucucaaccuaaccguugaga

SEQ ID NO: 15 - RNA, 17 nt, Bacteriophage P22
accgcccacaacgcggu

SEQ ID NO: 16 - RNA, 17 nt, Bacteriophage P22
ugcgcugacaaagcgcg

SEQ ID NO: 17 - RNA, 15 nt, Bacteriophage H19B
ucgcugacaaagcga

SEQ ID NO: 18 - RNA, 15 nt, Bacteriophage H19B
gcgcugacaaagcgc

SEQ ID NO: 19 - RNA, 15 nt, Bacteriophage HK97
ucgcugacaaagcga

SEQ ID NO: 20 - RNA, 15 nt, Bacteriophage HK97
gcggucacaaagcgc

SEQ ID NO: 21 - RNA, 15 nt, Bacteriophage 933W
ucgcugacaaagcga

SEQ ID NO: 22 - RNA, 15 nt, Bacteriophage 933W
gcgcugacaaagcgc

SEQ ID NO: 23 - RNA, 14 nt, Bacteriophage lambda
gccugaaaaagggc

SEQ ID NO: 24 - RNA, 14 nt, Bacteriophage lambda
gccuggaaaagggc

SEQ ID NO: 25 - RNA, 14 nt, Bacteriophage lambda
gccuguaaaagggc

SEQ ID NO: 26 - RNA, 14 nt, Bacteriophage lambda
gccugcaaaagggc

SEQ ID NO: 27 - RNA, 14 nt, Bacteriophage lambda
gccugagaaagggc

SEQ ID NO: 28 - RNA, 14 nt, Bacteriophage lambda
gccugaagaagggc

SEQ ID NO: 29 - RNA, 14 nt, Bacteriophage lambda
gccugaauaagggc

SEQ ID NO: 30 - RNA, 14 nt, Bacteriophage lambda
gccugaacaagggc

SEQ ID NO: 31 - RNA, 14 nt, Bacteriophage lambda
gccugaaagagggc

SEQ ID NO: 32 - RNA, 14 nt, Bacteriophage lambda
gccugaaauagggc

SEQ ID NO: 33 - RNA, 14 nt, Bacteriophage lambda
gccugaaacagggc

SEQ ID NO: 34 - RNA, 17 nt, Bacteriophage lambda
ggcccugaagaagggcu

SEQ ID NO: 35 - protein, 18 aa, Bacteriophage phi-21
GTAKSRYKARRAELIAER

SEQ ID NO: 36 - protein, 18 aa, Bacteriophage P22
GNAKTRRHERRRKLAIER

SEQ ID NO: 37 - protein, 22 aa, Bacteriophage lambda
MNAQTRRRERRAEKQAQWKAAN

SEQ ID NO: 38 - protein, 22 aa, Bacteriophage lambda
MNAKTRRRERRAEKQAQWKAAN

SEQ ID NO: 39 - protein, 22 aa, Bacteriophage lambda
MNARTRRRERRAEKQAQWKAAN

SEQ ID NO: 40 - protein, 22 aa, Bacteriophage lambda
GNARTRRRERRAEKQAQWKAAN

SEQ ID NO: 41 - protein, 22 aa, Bacteriophage lambda
GNARTRRRERRAMERATLPQVL

SEQ ID NO: 42 - protein, 21 aa, Bacteriophage lambda
DAQTRRRERRAEKQAQWKAAN

SEQ ID NO: 43 - protein, 21 aa, Bacteriophage lambda
AAQTRRRERRAEKQAQWKAAN

SEQ ID NO: 44 - protein, 21 aa, Bacteriophage lambda
DSQTRRRERRAEKQAQWKAAN

SEQ ID NO: 45 - protein, 21 aa, Bacteriophage lambda
DAQARRRERRAEKQAQWKAAN

SEQ ID NO: 46 - protein, 21 aa, Bacteriophage lambda
DAQTKRRERRAEKQAQWKAAN

SEQ ID NO: 47 - protein, 21 aa, Bacteriophage lambda
DAQTRRRARRAEKQAQWKAAN

SEQ ID NO: 48 - protein, 21 aa, Bacteriophage lambda
DAQTRRREKRAEKQAQWKAAN

SEQ ID NO: 49 - protein, 21 aa, Bacteriophage lambda
DAQTRRRERKAEKQAQWKAAN

SEQ ID NO: 50 - protein, 21 aa, Bacteriophage lambda
DAQTRRRERRGEKQAQWKAAN

SEQ ID NO: 51 - protein, 21 aa, Bacteriophage lambda
DAQTRRRERRAAKQAQWKAAN

SEQ ID NO: 52 - protein, 21 aa, Bacteriophage lambda
DAQTRRRERRAPKQAQWKAAN

SEQ ID NO: 53 - protein, 21 aa, Bacteriophage lambda
DAQTRRRERRAERQAQWKAAN

SEQ ID NO: 54 - protein, 21 aa, Bacteriophage lambda
DAQTRRRERRAEKQGQWKAAN

SEQ ID NO: 55 - protein, 21 aa, Bacteriophage lambda
DAQTRRRERRAEKQAAWKAAN

SEQ ID NO: 56 - protein, 21 aa, Bacteriophage lambda
DAQTRRRERRAEKQAQFKAAN

SEQ ID NO: 57 - protein, 21 aa, Bacteriophage lambda
DAQTRRRERRAEKQAQYKAAN

SEQ ID NO: 58 - protein, 21 aa, Bacteriophage lambda
DAQTRRRERRAEKQAQWAAAN

SEQ ID NO: 59 - protein, 21 aa, Bacteriophage lambda
DAQTRRRERRAEKQAQWKGAN

SEQ ID NO: 60 - protein, 21 aa, Bacteriophage lambda
DAQTRRRERRAEKQAQWKAGN

SEQ ID NO: 61 - protein, 21 aa, Bacteriophage lambda
DAQTRRRERRAEKQAQWKAAA

SEQ ID NO: 62 - protein, 18 aa, Artificial Sequence
  Synthesized
XXAXXRXXXRRXXXXXXX

SEQ ID NO: 63 - protein, 35 aa, Coliphage HK022
DSGQNRKVSDRGLTSRDRRRIARWEKRIAYALKNG

SEQ ID NO: 64 - DNA, 148 bp, Bacillus anthracis
tcatcacactttcacctttgtccactaagtcaccttaatgatttaagtaaaagtttgcggttgtgatgaacaaccgggagtcatccgccgaaatctcgataaaccccacctcgtcaactgagcccttcttcagggcccagttcggcgc

SEQ ID NO: 65 - DNA, 150 bp, Bacillus anthracis
agatcatgtgatttctctttgttcaaggagtcacccccttggtttgaagaaatccttacggctgtgatctgcacactcagccgggaggcatccgccgaaaattcgataacctccatcctcgtcaactaccggtaccggtagttcgggcgc

SEQ ID NO: 66 - DNA, 82 bp, Artificial Sequence
  Synthesized
gctttaatacgactcactatagcagattgagggtctatgataccacccggtacaggagatatctgcagcgccgaactgggcc

SEQ ID NO: 67 - DNA, 82 bp, Artificial Sequence
  Synthesized
ggcccagttcggcgctgcagatatctcctgtaccgggtggtatcatagaccctcaatctgctatagtgagtcgtattaaagc

SEQ ID NO: 68 - DNA, 24 bp, Artificial Sequence
  Synthesized
tcacacaggaaacagctatgacca

SEQ ID NO: 69 - RNA, 147 nt, Artificial Sequence
  Synthesized
gcgccgaacugggcccugaagaagggcucaguugacgaggugggguuuaucgagauuucggcggaugacucccgguuguucaucacaaccgcaaacuuuuacuuaaaucauuaaggugacuuaguggacaaaggugaaagugugaug

SEQ ID NO: 70 - RNA, 175 nt, Artificial Sequence
  Synthesized
gcgccgaacugguaccauugcacuccggugccaguugacgaggugggguuuaucgagauuucggcggaugacucccgguuguucaucacaaccgcaaacuuuuacuuaaaucauuaaggugacuuaguggacaaaggugaaagugugaugaccuugggcccugaagaagggcucg

SEQ ID NO: 71 - RNA, 198 nt, Artificial Sequence
  Synthesized
gcgccgaacugguaccauugcacuccggugccaguugacgaggugggguuuaucgagauuucggcggaugacucccgguuguucaucacaaccgcaaacuuuuacuuaaaucauuaaggugacuuaguggacaaaggugaaagugugaugaccuugggcccugaagaagggcucuucucucgcccugaagaagggcgg

SEQ ID NO: 72 - RNA, 147 nt, Artificial Sequence
  Synthesized
gcgccgaacugcgcgcagacaaugcgcguaguugacgaggugggguuuaucgagauuucggcggaugacucccgguuguucaucacaaccgcaaacuuuuacuuaaaucauuaaggugacuuaguggacaaaggugaaagugugaug

SEQ ID NO: 73 - RNA, 157 nt, Artificial Sequence
  Synthesized
gcgccgaacuggcuggcgcgcagacaaugcgcgucagccaguugacgaggugggguuuaucgagauuucggcggaugacucccgguuguucaucacaaccgcaaacuuuuacuuaaaucauuaaggugacuuaguggacaaaggugaaagugugaug

SEQ ID NO: 74 - RNA, 157 nt, Artificial Sequence
  Synthesized
gcgccgaacuggcuggggcccugaagaagggcuccagccaguugacgaggugggguuuaucgagauuucggcggaugacucccgguuguucaucacaaccgcaaacuuuuacuuaaaucauuaaggugacuuaguggacaaaggugaaagugugaug

SEQ ID NO: 75 - RNA, 147 nt, Artificial Sequence
  Synthesized
gcgccgaacugggcccugaagaagggcucaguugacgagguagacguuaucgagauuucggcggaugcgucucgguuguucaucacaaccgcaaacuuuuacuuaaaucauuaaggugacuuaguggacaaaggugaaagugugaug